(19) United States
(12) Patent Application Publication
(10) Pub. No.: US 2026/0224657 A1
(43) Pub. Date: Aug. 6, 2026

(54) Treatment Of Nicotinamide Phosphoribosyltransferase (NAMPT) Related Diseases

(71) Applicant: INSTITUTE FOR CANCER RESEARCH D/B/A THE RESEARCH INSTITUTE OF FOX CHASE CANCER, PHILADELPHIA, PA (US)

(72) Inventors: Johnvesly Basappa, Philadelphia, PA (US); Mariusz Wasik, Philadelphia, PA (US)

(73) Assignee: INSTITUTE FOR CANCER RESEARCH D/B/A THE RESEARCH INSTITUTE OF FOX CHASE CANCER, PHILADELPHIA, PA (US)

(21) Appl. No.: 19/468,690

(22) Filed: Feb. 3, 2026

Related U.S. Application Data
(60) Provisional application No. 63/753,178, filed on Feb. 3, 2025.

(51) Int. Cl. A61K 38/10; A61K 31/403; A61K 31/44; A61K 31/4545; A61K 31/506; A61K 31/55; A61P 35/00

(52) U.S. Cl. CPC A61K 38/10; A61K 31/403; A61K 31/44; A61K 31/4545; A61K 31/506; A61K 31/537; A61K 31/5386; A61P 35/00

(57) ABSTRACT

The disclosure generally relates to methods for the treatment of cancer by inhibiting Nicotinamide Phosphoribosyltransferase (NAMPT) and to methods of treating diabetes, obesity, and neurodegenerative diseases by activating NAMPT.

### PRIORITY CLAIM AND CROSS-REFERENCED TO RELATED APPLICATION

This application claims the benefit of U.S. Provisional Application No. 63/753,178, filed Feb. 3, 2025, which application is expressly incorporated by reference herein in its entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Feb. 3, 2026, is named J4074-00003_SL.xml, and is 19,576 bytes in size.

### FIELD

The disclosure generally relates to compositions and methods for the treatment of cancer by inhibiting Nicotinamide Phosphoribosyltransferase (NAMPT) and to compositions and methods of treating metabolic disorders including diabetes, obesity, and neurodegenerative diseases by activating NAMPT.

### BACKGROUND

In most cancers, diverse tyrosine kinases play an important role in disease pathogenesis and progression by regulating key cell-signaling mediators and metabolic enzymes by post-translational modifications. The total tyrosine phosphorylation of proteins accounts for only 2.5% of their amino-acid structure but confers a profound impact on their enzymatic activity and, ultimately, on diverse cell functions (Hunter, Curr. Opin. Cell. Biol., 2009, 21, 40-146). It has been demonstrated that tyrosine kinase-mediated phosphorylation of key metabolic enzymes such as ACLY (Basappa et al., Heliyon, 2020, 6), hexokinases HK1 and HK2 (Zhang et al., Nat. Commun., 2017, 8), Pyruvate Kinase M2 (PKM2) (Christofk et al., Nature, 2008, 452, 181-U127), Pyruvate Dehydrogenase Kinase (PDK1) (Hitosugi et al., Mol. Cell., 2011, 44, 864-877) and Lactate Dehydrogenase (LDHA) (Fan et al., Mol. Cell. Biol., 2011, 31) regulates tumor metabolism.

Anaplastic lymphoma kinase (ALK) was initially discovered as an oncogene in human anaplastic large cell lymphomas by gene fusion of ALK with NPM in (ALCL) (Morris et al., Science, 1994, 263, 1281-1284) and EML4 in non-small cell lung cancer (NSCLC) (Soda et al., Nature, 2007, 448, 561-566), together with LTK and ROS, ALK belongs to the insulin receptor superfamily of cell membrane-spanning receptors that display intrinsic tyrosine kinase activity (Werner et al., Blood, 2017, 129, 823-831). Anaplastic large cell lymphoma (ALCL) is an aggressive subtype of non-Hodgkin lymphoma representing 3% of adult and 10-15% of pediatric non-Hodgkin lymphomas (Swerdlow et al., WHO Classification of Tumours of Haematopoietic and Lymphoid Tissues, 2017, IARC). NPM-ALK is a potent oncogene and its ectopic expression in vitro and in vivo using transgenic models results in oncogenesis (Pawlicki et al., Cancer Res., 2021, 81, 3241-3254; and Zhang et al., Am. J. Pathol., 2013, 183, 1971-1980). Furthermore, ALK fusions are oncogenic in other cancers including non-small cell lung cancer (Soda et al., Nature, 2007, 448, 561-566), inflammatory myofibroblastic tumor (Butrynski et al., N. Engl. J. Med., 2010, 363, 1727-1733), and mutations of full length ALK play a role in pathogenesis of neuroblastoma (Chen et al., Nature, 2008, 455, 971-974).

NAMPT as a rate-limiting enzyme, converts nicotinamide (NAM) a form of water-soluble vitamin B3 to nicotinamide mononucleotide (NMN) (Yoshino et al., Cell. Metab., 2018, 27, 513-528), which is then adenylated to nicotinamide adenine dinucleotide (NAD) by nicotinamide mononucleotide adenylyl transferase (NMNATs) (Sharif et al., Antioxid Redox Sign., 2019, 30, 906-923). In mammalian cells, NAD is synthesized through three different pathways such as de novo synthesis from tryptophan, from nicotinic acid (NA) using the Preiss Handler Pathway (PH) and synthesis from nicotinamide (NAM) or nicotinamide riboside (NR) via the salvage-pathway by three rate-limiting enzymes of quinolinate phosphoribosyltransferase (QAPRT), nicotinate phosphoribosyltransferase (NAPRT) and nicotinamide phosphoribosyltransferase (NAMPT), respectively (Chowdhry et al., Nature, 2019, 569, 570; Yaku et al., Front Oncol., 2018, 8; Xie et al., Signal Transduct. Tar., 2020, 5; Imai, Curr. Pharm. Design, 2009, 15, 20-28; Verdin, Science, 2015, 350, 1208-1213; Chiarugi et alo., Nat. Rev. Cancer, 2012, 12, 741-752; Piacente et al., Cancer Res., 2017, 77, 3857-3869). The main source of NAD synthesis in higher vertebrates comes from nicotinamide (NAM) by using NAMPT enzyme (Li et al., Front Oncol., 2019, 9; and Dierickx et al., Nat. Cardiovasc. Res., 2022, 1, 45-58).

Human NAMPT is a 55-kDa enzyme, made of 491 amino acids that form a homodimer to generate two active sites comprising catalytic residues knowh as H247 (also known as His27). NAMPT is a dimer in cellular conditions and is essential for NAD biosynthesis. NAMPT exists in two forms: intracellular (iNAMPT), which mainly exists as a dimer, and extracellular (eNAMPT), which exists as a monomer.

NAMPT is overexpressed in many cancers such as prostate (Wang et al., Oncogene, 2011, 30, 907-921), glioma (Lucena-Cacace et al., Oncotarget, 2017, 8, 99514-99530), melanoma (Audrito et al., Cancers, 2020, 12), lung (Okumura et al., J. Thorac. Oncol., 2012, 7, 49-56), colon (Lucena-Cacace et al., Clin. Cancer Res., 2018, 24, 1202-1215; and Ye et al., Cell. Commun. Signal, 2020, 18, 16), breast (Zhou et al., Oncol. Lett, 2018, 15, 6648-6654; Pour et al., Bmc Cancer, 2019, 19; and Bajrami et al., Embo Mol. Med., 2012, 4, 1087-1096), thyroid (Sawicka-Gutaj et al., Tumor Biol., 2015, 36, 7859-7863), kidney (Abu Aboud et al., Mol. Cancer Ther., 2016, 15, 2119-2129), and pancreatic cancer (Moore et al., Proc. Natl Acad. Sci. USA, 2021, 118; Ju et al., Cancer Lett., 2016, 379, 1-11; Moore et al., Cell Death Dis., 2015, 6). NAMPT has 27 tyrosine residues, of which 13 of tyrosine phosphorylated residues were documented in phosphosite.org, and the remaining 8 residues were predicted by Phosphonet.ca human phosphosite knowledge basewebsite to be NAMPT phosphorylation sites. What is lacking, however, is experimental evidence on the direct link between tyrosine kinase and NAMPT phosphorylation. Since, many metabolic enzyme activities are regulated by tyrosine kinases through tyrosine phosphorylation, it is necessary to better understand the mechanics and dynamics of NAMPT tyrosine phosphorylation as well as resulting functions and activities.

As previously discussed, due to abnormal proliferation and higher energy demand, tumor cells are more dependent on NAD+ than normal cells. Given the key role played by NAMPT in the biosynthesis pathway for NAD+, inhibitors ofNAMPT may provide potentially therapeutic components for the treatment of cancer and other such malignanicies.

The role of NAMPT is widespread phsiologically, and in certain disease states such a metabolic disorders, it may be beneficial to increase the level of NAD+ and as such activators of NAMPT may provide potentially therapeutic components for the treatment of diabetes, obesity, inflammation, heart disease, liver disease as well neurodegenerative disease (motor neuron disease (ALS), sensory-motor neuropathy (MINA syndrome)), and broader issues related to aging.

What is needed is a comprehensive understanding of the mechanisms by which NAMPT is inhibited and activated. Such an understanding will enable the development of useful inhibitors and activators thereby providing useful therapeutic components that may be utilized for the treatment of a variety of abnormal conditions ranging from cancer to metabolic and neurodegenerative disorders. Currently, such inhibitors and/or activators with sufficient efficacy and minimal toxicity are unavailable.

### SUMMARY

The present disclosure demonstrates the role of oncogenic tyrosine kinases in the phosphorylation of NAD+-synthesizing enzyme NAMPT, focusing on its impact on enzyme activity and tumor metabolism in hematological malignancies as well as other metabolic disorders.

Using in-vitro kinase assays, the inventors demonstrated that various oncogenic tyrosine kinases, including ALK, IR, IGF1R, BTK, and SYK, directly phosphorylate NAMPT at the Y188 residue, significantly increasing enzyme activity. Comprehensive phosphoproteomics analysis revealed ten tyrosine phosphorylation sites on NAMPT, with along with additional novel findings. The mutation of NAMPT Y188 specific tyrosine residues to phenylalanine (Y188F) highlighted the critical role of these residues in regulating NAMPT activity. Further studies on novel synthesized peptides derived from the NAMPT phospho-domain surrounding the phosphorylated tyrosine demonstrated their inhibitory effects on NAMPT activity.

The inventors herein have elucidated novel aspects of NPM-ALK and dimeric NAMPT translocation in the mitochondria of ALCL cell line: the translocation of the monomeric form of NAMPT to the mitochondria is reduced and the translocation of NAMPT and its impact on mitochondrial function are kinase dependent. The present disclosure provides a novel perspective on the metabolic regulatory function of NAMPT tyrosine phosphorylation and reveals that NPM-ALK plays an important role in the regulation of cellular metabolism and cancer cell resistance to therapeutics. As shown herein, ALK inhibition significantly decreased levels of NMN and its end product, NAD+, in LC-MS/MS targeted metabolite analysis of ALK+ALCL cells treated with ALK inhibitor certinib. Furthermore, the inventors evaluated the effects of NAMPT phosphorylation on cell proliferation by culturing stable SUPM2 cell lines expressing various NAMPT constructs. The inventors observed a notable reduction in cell proliferation in NAMPT Y188F mutants compared to wild-type NAMPT. Clonogenic assays revealed that wild-type NAMPT conferred a significant advantage in colony formation. The inventors further demonstrate that NPM-ALK and other tyrosine kinases phosphorylate NAMPT Y188, facilitating NMN and NAD+ biosynthesis and enhancing cellular proliferation, highlighting the importance of NAMPT in ALK-driven cancers.

The present disclosure provides novel compositions and methods for the treatment of abnormal conditions involving the inhibiting or activating of NAMPT.

The present disclosure provides novel compositions and methods for the treatment of abnormal conditions involving the inhibiting or activating of NAMPT wherein such novel peptides and compositions thereof that are nontoxic and display little or no adverse effects.

The present disclosure provides methods of treating cancer in a subject in need thereof comprising administering to the subject an NAMPT inhibitor.

The present disclosure further provides methods of diagnosing a cancer in a subject comprising detecting the presence of Y188 tyrosine phosphorylation of NAMPT in a cell obtained from the subject.

The present disclosure also provides a peptide comprising the amino acid sequence

(SEQ ID NO: 1)
KLHDFGYRGVSSQE.

The present disclosure provides methods of treating diabetes, obesity, inflammation, heart disease, liver disease as well neurodegenerative disease (motor neuron disease (ALS), sensory-motor neuropathy (MINA syndrome)), and broader issues related to aging in a subject in need thereof comprising administering to the subject an NAMPT activator or NAMPT inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides data related to oncogenic tyrosine kinases directly phosphorylating NAMPT on tyrosine residues. FIG. 1 (Panel A) provides a schematic of the hypothesis showing various tyrosine kinases directly phosphorylating NAMPT Y34 and Y188 residues. FIG. 1 (Panel B) provides an immunoblot demonstrating that tyrosine kinases directly phosphorylate NAMPT on tyrosine residues. In-vitro kinase assay on recombinant NAMPT in the presence of active tyrosine kinases. FIG. 1 (Panel C) provides a schematic of in-vitro kinase assay; Purified recombinant human NAMPT and active BTK and SYK kinase in the presence of ATP and kinase buffer. FIG. 1 (Panel D) provides a graph showing densitometry values of p-NAMPT (p-Tyrosine) vs. NAMPT from three independent experiments. FIG. 1 (Panel E) provides a graph showing the results of a NAMPT activity assay: In-vitro phosphorylated NAMPT has increased enzyme activity. FIG. 1 (Panel F) provides an immunoblot showing in-vitro kinase samples Immunoblotted and probed for the indicated antibodies: Spleen tyrosine kinase (SYK) and Bruton tyrosine kinase (BTK) directly phosphorylate NAMPT on Tyrosine residues. FIG. 1 (Panel G) provides an immunoblot in-vitro kinase samples resolved on Native PAGE gel and stained with colloidal blue for LC/MS/MS analysis. FIG. 1 (Panel H) provides a table illustrating BTK and SYK mediated NAMPT tyrosine phosphorylated peptides identified in phosphoproteomics by LC/MS/MS analysis: Y34 (SEQ ID NO: 2), Y54 (SEQ ID NO: 3), Y87 (SEQ ID NO: 4), Y103 (SEQ ID NO: 5), Y108 (SEQ ID NO: 6), Y1188 (SEQ ID NO: 7), Y195 (SEQ ID NO: 8), Y341 (SEQ ID NO: 9), Y453 (SEQ ID NO: 10), andY471 (SEQ ID NO: 11).

FIG. 1 (Panel I) provides a schematic representation of human NAMPT phosphorylation sites identified in LC/MS/MS. * Indicates first-time reports in this study. The PRISM GraphPad software was used to analyze t-test (p>value; 0.05*, 0.005***0.0005****)

FIG. 2 provides data related to ALK regulation of tyrosine phosphorylation of NAMPT in ALK+ALCL lymphoma. FIG. 2 (Panel A) provides an immunoblot demonstrating that Immunoblotting of p-NAMPT Y188 on NPM-ALK positive ALCL ALK-negative lymphoma and mantle cell lymphoma cell lines. FIG. 2 (Panel B) provides a histograph showing IHC staining ALK+ALCL patient biopsy samples. FIG. 2 (Panel C) provides a schematic of an immunoblot ALKi certinib treated SUDHL-1 and SUP-M2 cell lysates. FIG. 2 (Panel D) provides a schematic of an immunoblot ALKi certinib treated human NA1 (CD4+ cells transduced with NPM-ALK) cell lysates Immunoblotting.

FIG. 3 provides data demonstrating that oncogenic ALK R1275Q mutated in neuroblastoma (NB) and Insulin receptor (IR) increases NAMPT phosphorylation and enzyme activity. FIG. 3 (Panel A) provides an immunoblot demonstrating In-vitro kinase assay on recombinant NAMPT with ALK, ALK R1275Q, and human insulin receptor (IR), tyrosine kinases and immunoblotted with p-NAMPT Y188 and HA (for NAMPT) antibodies. FIG. 3 (Panel B) provides a graph showing densitometry analysis of the immunoblot from FIG. 3 (Panel A) and other two independent blots showing p-NAMPT Y188/NAMPT normalized values (A.U). FIG. 3 (Panels B & C) provide graphs showing NAMPT activity assay on two independent samples. FIG. 3 (Panel D) provides an immunoblot showing the results of HEK-293T stable cell line NAMPT-WT and NAMPT-Y188F expression when transiently transfected with ALK, ALK R1275Q, and human insulin receptor (IR) and SYK, tyrosine kinases lysates as Immunoblotted with p-NAMPT Y188 and HA (for NAMPT) antibodies. FIG. 3 (Panel E) provides an immunoblot showing the results of HEK-293T stable cell line NAMPT-WT, NAMPT-Y34F, and NAMPT-Y188F expressing when transiently transfected with IR, IGF1R, and HER2 tyrosine kinases lysates as Immunoblotted with p-NAMPT, Y188 and HA (for NAMPT) antibodies. FIG. 3 (Panel F) provides an immunoblot showing the results of NAMPT-WT and IR, IFG1R expressing cell lysates. FIG. 3 (Panel G) provides a graph showing densitometry analysis of the (FIG. 3, Panel F) immunoblot and other two independent blots showing p-NAMPT Y188/NAMPT normalized values (A.U). FIG. 3 (Panel H) provides a graph showing densitometry analysis NAMPT activity assay on two independent samples. The PRISM GraphPad software was used to analyze t-test (p>value; 0.05*, 0.005***0.0005****)

FIG. 4 provides data demonstrating that dimerization of NAMPT is critical for NAMPT tyrosine phosphorylation on 188 residues. FIG. 4 (Panel A) provides an immunoblot showing ALK+ALCL, SUPM2 cell lines stably expressing NAMPT-WT, NAMPT-Y188F and NAMPT-SS-AA (dimer breaking interface) cell lysates as immunoblotted with p-NAMPT Y188, HA (NAMPT), ALK and GAPDH. FIG. 4 (Panel B) provides a graph showing densitometry analysis of the FIG. 4 (Panel A) immunoblot and other 2 independent blots showing p-NAMPT Y188/NAMPT normalized values (A.U). FIG. 4 (Panel C) provides a graph showing NAMPT activity assay using the samples from 2 independent samples. FIG. 4 (Panel D) provides an immunoblot showing ALK+ALCL, SUDHL1 cell lines stably expressing NAMPT-WT, NAMPT-Y188F and NAMPT-SS-AA (dimer breaking interface) cell lysates as immunoblotted with p-NAMPT Y188, HA (NAMPT), ALK and GAPDH. FIG. 4 (Panel E) provides a graph showing densitometry analysis of the FIG. 4 (Panel D) immunoblot from two independent blots showing p-NAMPT Y188/NAMPT normalized values (A.U). FIG. 4 (Panel F) provides a graph showing NAMPT activity assay using the samples from two independent samples. FIG. 4 (Panels G & H) provide immunoblots of ALK+ALCL, SUPM2 cell lines stably expressing NAMPT-WT, NAMPT-Y188F, and NAMPT-SS-AA (dimer-breaking interface) cell lysates and 293T expressing ACLY-HA (positive control) when separated on non-reducing conditions, native-PAGE and immunoblotted with the HA and phospho-NAMPT Y188 antibodies. FIG. 4 (Panels I & J) provide immunoblots of ALK+ALCL and SUPM2 cell lines stably expressing NAMPT-WT, NAMPT-Y188F, and NAMPT-SS-AA cell lysates when immunoprecipitated with HA antibody, separated on SDS-PAGE, and immunoblotted with the HA and phospho-NAMPT Y188 antibodies. FIG. 4 (Panels K & L) provide immunoblots showing ALK+ALCL and SUPM2 cell lines stably expressing NAMPT-WT, NAMPT-Y188F, and NAMPT-SS-AA cell lysates when immunoprecipitated with the HA antibody, separated on SDS-PAGE, and immunoblotted with ALK and phospho-ALK Y1604 antibodies. The PRISM GraphPad software was used to analyze the t-test (p>value; 0.05*, 0.005***0.0005****).

FIG. 5 provides data demonstrating that dimerization of NAMPT is critical for mitochondrial and nuclear translocation. FIG. 5 (Panel A) provides an immunoblot showing the results of an experiment wherein SUDHL-1 cell lines stably expressing NAMPT-WT, NAMPT-Y188F, and NAMPT-SS-AA cells were subjected to cytosolic and mitochondrial fractionation, and lysates were separated on SDS-PAGE and immunoblotted with the indicated antibodies. FIG. 5 (Panel B) provides an immunoblot showing the results of an experiment wherein SUDHL-1 cell lines stably expressing NAMPT-WT, NAMPT-Y188F, and NAMPT-SS-AA cells were subjected to cytosolic and nuclear fractionation, and lysates were separated on SDS-PAGE and immunoblotted with the indicated antibodies. FIG. 5 (Panel C) provides a schematic overview of NAMPT translocation in various compartments.

FIG. 6 provides data demonstrating that NAMPT tyrosine phosphorylation regulates NMN and NAD biosynthesis and cell growth in cancer. FIG. 6 (Panel A) provides a schematic illustrating how the ALK inhibitor, ceritinib, affects NAMPT phosphorylation and the biosynthesis of NMN and NAD+. FIG. 6 (Panel B) provides a graph showing the effect of certinib on NMN metabolite in SUPM2 FIG. 6 (Panel C) provides a graph showing the effect of certinib on NAD metabolite in SUPM2 FIG. 6 (Panel D) provides a graph showing SUDH-L1 stable cell lines expressing GFP tagged NAMPT-WT, NAMPT-Y188F, and NAMPT-SS-AA and their cell growth measured by GFP fluorescence. FIG. 6 (Panel E) provides a photograph of methylcellulose colony formation assay on SUDHL1 stable cell lines. FIG. 6 (Panel F) provides a graph of showing the quantitation of the colony in FIG. 6 (Panel E) by ImageJ software. FIG. 6 (Panel G) provides a graphical summary of the findings shown in FIG. 6 Panels A-F. The PRISM GraphPad software was used to analyze the t-test (p>value; 0.05*, 0.005***0.0005****).

FIG. 7 provides data demonstrating that NAMPT Tyrosine-domain peptides inhibit NAMPT activity in-vitro activity assay. FIG. 7 (Panel A) provides sequences of NAMPT tyrosine phosphorylated residues and peptides Y34 (SEQ ID NO: 2), Y54 (SEQ ID NO: 3), Y103 (SEQ ID NO: 5), Y108 (SEQ ID NO: 6), Y195 (SEQ ID NO: 8), and Y453 (SEQ ID NO: 10).

FIG. 7 (Panel B) provides a graph showing the activity of the NAMPT peptides used in NAMPT activity assay. FIG. 7 (Panel C) provides data showing that the sequence of NAMPT Y188 is highly conserved in various species. FIG. 7 (Panel D) provides a pictograph showing the dimer interface of NAMPT in the proximity of Y188 and Y195 residues.

FIG. 8 provides data showing that NAMPT is tyrosine phosphorylated by SYK, BTK, and SRC kinases. FIG. 8 (Panel A) provides an immunoblot showing the results of an assay wherein stable HEK-293 T cell line NAMPT-WT and NAMPT-Y188F expressing cells were transiently transfected with SYK, BTK, and SRC and treated with respective TKI. The lysates were immunoblotted with p-NAMPT, Y188, and HA (for NAMPT) antibodies. FIG. 8 (Panel B) provides a graphical summary of tyrosine kinase-mediated NAMPT phosphorylation and TKI role.

FIG. 9 provides a data showing the computational and molecular docking of NAMPT Y188 phosphorylation domain containing seven amino acids peptide binds to a critical lysine 1150 (K1150) residue on human ALK FIG. 9 (Panel A) shows the crystal structure of ALK. FIG. 9 (Panel B) provides a schematic of a linear peptide of NAMPT and its structure. FIG. 9 (Panel C) provides a schematic of ALK and NAMPT peptide docking. FIG. 9 (Panel D) provides ALK and NAMPT peptide docking showing K1150 and V1130 residues.

FIG. 10 provides data demonstrating that NPM-ALK directly phosphorylates the critical NAMPT residue Y188 and regulates NAMPT dimerization. FIG. 10 (Panel A) provides a schematic of phosphoproteomics workflow describing NAMPT-HA-tagged wild-type (dimer) and monomer (SS_AA) stable cell line cultures, followed by HA immunoprecipitation and LC-MS/MS analysis; FIG. 10 (Panel B) provides results of lysates subjected to Western blotting using the p-NAMPT Y188 antibody to confirm NAMPT phosphorylation in NPM-ALK-transfected cell lysates; FIG. 10 (Panel C) provides a heatmap showing NAMPT Y188, the major tyrosine residue, with spectral intensity in NAMPT-WT and NAMPT SS_AA mutant cells transfected with active NPM-ALK.K; FIG. 10 (Panel D) provides the NAMPT Y residue spectral intensity of NAMPT-WT (dimer) and monomer; FIG. 10 (Panel E) provides NAMPT tyrosine phosphorylated residue sequences Y34 (modified) (SEQ ID NO: 18), Y175 (SEQ ID NO: 12), Y188 (modified) (SEQ ID NO: 19), and Y403 (SEQ ID NO: 13); and FIG. 10 (Panel F) provides a schematic of NAMPT Y residues.

### DESCRIPTION OF EMBODIMENTS

Various terms relating to aspects of the disclosure are used throughout the specification and claims. Such terms are to be given their ordinary meaning in the art, unless otherwise indicated. Other specifically defined terms are to be construed in a manner consistent with the definition provided herein. Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by l10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the terms “individual,” “subject,” and “patient,” used interchangeably, mean any animal described herein.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor, or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects.

Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

NAMPT is a critical rate limiting enzyme responsible for direct biosynthesis of NMN, a crucial product that is later directly synthesized into NAD+ by nicotinamide/nicotinic acid mononucleotide adenylyl transferase (NMNAT) enzyme in salvage pathways. This pathway is common in both normal and cancer cells. However, cancer cells overexpress NAMPT and are enzymatically very active due to tyrosine phosphorylation by oncogenic tyrosine kinase. Y188, also known as NAMPT Y188 and Tyrosine 188, is a key amino acid residue located in the catalytic channel of NAMPT and is often involved with the interaction of molecules that bind NAMPT. The inventors herein utilized biochemical and genetic approaches to determine the role of phosphorylation of NAMPT Y188 in regulation of its enzymatic activity in NAD metabolism. For the first time, with the use of tools such as in vitro kinase assays, the inventors demonstrated that a variety of oncogenic kinases directly phosphorylate NAMPT. Also, for the first time, this inventors demonstrate that insulin receptor and ALK oncogenic kinases directly phosphorylate NAMPT on Y188 residue using both in vitro and in vivo cellular systems. In ALCL, NPM-ALK mediated tyrosine phosphorylation of NAMPT regulates production of NMN from nicotinamide, the rate limiting step in the generation of NAD. ALK+ALCL lymphoma expressed higher levels of NAMPT and p-NAMPT Y188 than mantle cell lymphoma cell lines.

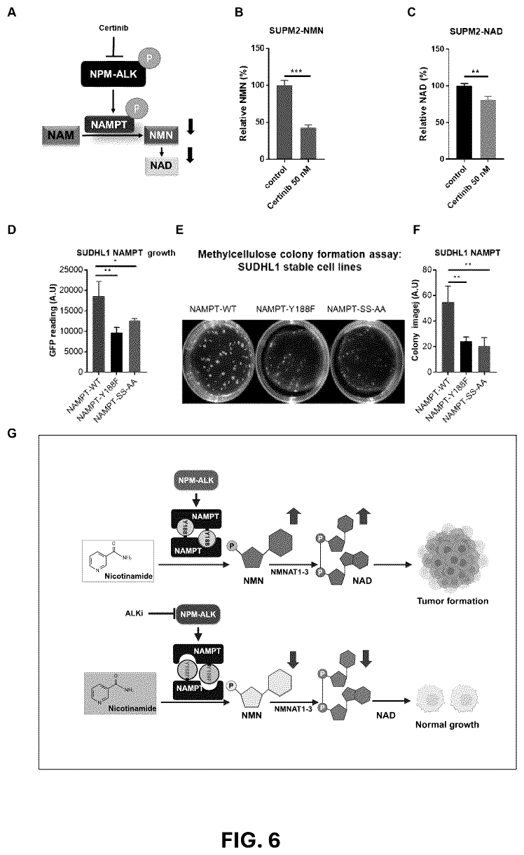

The gene encoding wild-type ALK, as well as well-characterized activating mutations in full-length ALK (R1275Q) in neuroblastoma, phosphorylates NAMPT at Y188 more than wild-type ALK and overregulates enzyme activity: the mutant ALK targets its downstream substrate with higher affinity for phosphorylation than nonmutant ALK. In addition, insulin receptor and IGF1R tyrosine kinases also phosphorylate NAMPT at Y188. Though not wishing to be bound by the following, the inventors proposed that constitutive phosphorylation of NAMPT Y188 by oncogenic tyrosine kinase NPM-ALK or insulin receptor or other physiological stimulations, act as an NAMPT activator to increase the NMN availability for NAD+ biosynthesis. Additionally, as shown in the Examples, the inventors demonstrated that the tyrosine phosphorylation of NAMPT enhances its enzyme activity, allowing it to convert the substrate nicotinamide (NAM) into nicotinamide mononucleotide (NMN) in the presence of the insulin family receptor, anaplastic lymphoma kinase (ALK). However, the synthesis of NMN was impaired when ALK function was inhibited using the small molecule inhibitor certinib (FIG. 6). In this context, the phosphorylation of NAMPT by tyrosine kinases such as Insulin receptor (IR) and IGF1R plays an important role in normal physiology and maintaining healthy glucose homeostasis and preventing neurodegenerative disease by increasing the synthesis of NMN.

The inventors investigated and demonstrated that boosting NAD+ level through tyrosine kinase mediated NAMPT phosphorylation and activation is an attractive approach for countering the effects of metabolic disease and aging. The inventors' study in ALK+ALCL lymphoma, demonstrated that NAMPT is constitutively active due to oncogenic NPM-ALK tyrosine kinase. Moreover, the observation that physiologic growth stimuli such as insulin and insulin receptor axis mediated phosphorylation of NAMPT may serve as a switch that directly controls the ability of critical cellular events to immediately trigger NMN and NAD+ biosynthesis required for adipose tissue function, glucose homeostasis, insulin sensitivity and cell proliferation. The highly conserved NAMPT Y188 among all mammalians also supports the notion that this tyrosine phosphorylation site is a significant mode of regulation of its enzymatic activity.

The inventors also discovered a role of dimerization in tyrosine phosphorylation. They hypothesized and demonstrated that tyrosine phosphorylation enhances the dimerization of NAMPT and increases its activity. The marked reduction of NAMPT activity, significantly decreased phosphorylation in monomeric NAMPT, impaired cell proliferation and clonogenic ability in Y188F mutants in NPM-ALK+ cell line background shows that the phosphorylation at this residue is highly critical for oncogenesis, and direct inhibition of NAMPT Y188 phosphorylation or other mechanisms to disrupt its enzymatic activity provides an attractive therapeutic opportunity for ALK+ALCL and other ALK driven cancers. In summary, disclosed herein is the identification of NAMPT as a target of oncogenic tyrosine kinases, which have important implications for understanding tumor metabolism, including with regard to hematological malignancies. Based on the novel understanding that tyrosine phosphorylation enhances NAMPT activity, the inventors have designed and developed unique peptides to constitute therapeutic strategies that inhibit this pathway in cancer treatment. Furthermore, in discovering heretofore unknown specific phosphorylation sites on NAMPT, enables the understanding the NAMPT's regulatory mechanisms. The development of peptides that inhibit NAMPT activity highlights a unique avenue for drug development, as well as targeting the phospho-domains associated with enzyme regulation.

The present disclosure provides compositions and methods of treating cancer in a subject in need thereof comprising administering to the subject a Nicotinamide Phosphoribosyltransferase (NAMPT) inhibitor or NAMPT activator.

In some embodiments, the NAMPT inhibitor is a peptide. In some embodiments, the amino acid sequence of the peptide comprises PNTSKVYSYFECR (Pep-1; SEQ ID NO: 14), KLRKVKYEETVF (Pep-2; SEQ ID NO: 15), GWNYILEKYDGHL (Pep-3, SEQ ID NO: 16), KLHDFGYRGVSSQE (Pep-4, SEQ ID NO: 1), or KGDLEEYGQDL (Pep-5, SEQ ID NO: 17). In some embodiments, the amino acid sequence of the peptide comprises PNTSKVYSYFECR (Pep-1; SEQ ID NO: 14), GWNYILEKYDGHL (Pep-3, SEQ ID NO: 16), or KLHDFGYRGVSSQE (Pep-4, SEQ ID NO: 1). In some embodiments, the amino acid sequence of the peptide comprises PNTSKVYSYFECR (Pep-1; SEQ ID NO: 14). In some embodiments, the amino acid sequence of the peptide comprises KLRKVKYEETVF (Pep-2; SEQ ID NO: 15). In some embodiments, the amino acid sequence of the peptide comprises GWNYILEKYDGHL (Pep-3, SEQ ID NO: 16). In some embodiments, the amino acid sequence of the peptide comprises KLHDFGYRGVSSQE (Pep-4, SEQ ID NO: 1). In some embodiments, the amino acid sequence of the peptide comprises KGDLEEYGQDL (Pep-5, SEQ ID NO: 17).

TABLE 1
BTK AND SYK Kinases Directly Phosphorylate NAMPT on Tyrosine Kinases

| | Modifi- | | XCorr | |
|---|---|---|---|---|
| Annotated Sequence | cations | q-value | value | SEQ ID NO. |
| QYPPNTSKVYSYFECR | Y34 | 0.00000692 | 3.36 | SEQ ID NO: 2 |
| KVKYEETVFYGLQYILN | Y54 | 0.002176 | 3.66 | SEQ ID NO: 3 |
| K | | | | |
| IQEAKDVYKEHFQDDVF | Y87* | 0.0000494 | 3.1 | SEQ ID NO: 4 |
| NEK | | | | |
| GWNYILEK | Y103* | 0.01243 | 1.88 | SEQ ID NO: 5 |
| YDGHLPIEIK | Y108* | 0.0002001 | 3.63 | SEQ ID NO: 6 |
| YLLETSGNLDGLEYKLH | Y188 | 0.0002624 | 3.52 | SEQ ID NO: 7 |
| DFGYR | | | | |
| LHDFGYRGVSSQETAGI | Y195 | 1.163E−13 | 5.59 | SEQ ID NO: 8 |
| GASAHLYNFK | | | | |
| KFPVTENSKGYK | Y341* | 0.000003052 | 3.42 | SEQ ID NO: 9 |
| GDLEEYGQDLLHTVFK | Y453 | 4.133E−07 | 3.27 | SEQ ID NO: 10 |
| SYSFDEIR | Y471* | 0.002022 | 2.27 | SEQ ID NO: 11 |

In some embodiments, the NAMPT inhibitor is a small molecule. In some embodiments, the small molecule is daporinad (also known as FK866 and AP0866), GMX1777 (also known as EB1627), GMX1778 (also known as CHS828), KPT-9274 (also known as ATG-019), or OT-92. In some embodiments, the small molecule is daporinad. In some embodiments, the small molecule is GMX1777. In some embodiments, the small molecule is GMX1778. In some embodiments, the small molecule is KPT-9274. In some embodiments, the small molecule is OT-92.

In some embodiments, the NAMPT inhibitor is a compound that disrupts, interferes with, or prevents NAMPT dimerization and/or NAMPT phosphorylation. In some embodiments, the NAMPT inhibitor is a compound that disrupts, interferes with, or prevents NAMPT dimerization. In some embodiments, the NAMPT inhibitor is a compound that disrupts, interferes with, or prevents NAMPT phosphorylation.

In some embodiments, the cancer is ALK+ anaplastic large cell lymphoma (ALCL), EML4-ALK positive non-small cell lung cancer (NSCLC), or breast cancer with the activated form of the type 1 insulin-like growth factor receptor (IGF1R). In some embodiments, the cancer is ALK+ALCL. In some embodiments, the cancer is EML4-ALK positive NSCLC. In some embodiments, the cancer is breast cancer with the activated form of IGF1R.

In some embodiments, the methods further comprise administering a second therapeutic agent selected from the group consisting of an anaplastic lymphoma kinase (ALK) inhibitor, a bruton tyrosine kinase (BTK) inhibitor, a spleen tyrosine kinase (SYK) inhibitor, or any combination thereof. In some embodiments, the methods further comprise administering an ALK inhibitor. In some embodiments, the ALK inhibitor is certinib. In some embodiments, the methods further comprise administering a BTK inhibitor. In some embodiments, the methods further comprise administering an SYK inhibitor. In some embodiments, a combination of, for example, certinib and daporinad can be administered.

The compounds and pharmaceutical compositons described herein can be administered to a patient in need thereof in an oral formulation, an intravenous formulation, a topical formulation, an intraperitoneal formulation, an intrapleural formulation, an intravesical formulation, or an intrathecal formulation. The compositions may be formulated in a suitable controlled-release vehicle, with an adjuvant, or as a depot formulation. In some embodiments, the NAMPT inhibitor is administered to the subject intravenously.

The present disclosure also provides methods of diagnosing a cancer in a subject comprising detecting the presence of Y188 tyrosine phosphorylation of NAMPT in a cell obtained from the subject. In some embodiments, the cancer is ALCL, non-Hodgkin's lymphoma, pediatric non-Hodgkin's lymphoma, ALK+ anaplastic large cell lymphoma (ALCL), EML4-ALK positive non-small cell lung cancer (NSCLC), or breast cancer with the activated form of the type 1 insulin-like growth factor receptor (IGF1R). In some embodiments, the cancer is ALK+ALCL. In some embodiments, the cancer is EML4-ALK positive NSCLC.

In some embodiments, the cancer is breast cancer with the activated form of IGF1R. In some embodiments, detection of Y188 tyrosine phosphorylated NAMPT is carried out from a patient biopsy sample using, for example, anti-phospho-NAMPT Y188 antibody in an immunohistochemistry assay or in a phosphoproteomics analysis. In some embodiments, the cell obtained from the subject is a cell from a lymph node, lung, or breast.

The present disclosure also provides a peptide comprising the amino acid sequence KLHDFGYRGVSSQE (Pep-4, SEQ ID NO: 1). In some embodiments, the peptide is modified to contain a non-natural component such as, for example, an isotope, a label (such as a fluorescent label), or a heterologous tag (such as an antibody, or other protein or small molecules linked thereto).

The present disclosure also provides methods of treating diabetes, obesity, or a neurodegenerative disease in a subject in need thereof comprising administering to the subject an NAMPT activator. In some embodiments, diabetes is treated. In some embodiments, obesity is treated. In some embodiments, a neurodegenerative disease is treated. In some embodiments, the NAMPT activator is SBI-797812 or P7C3 (both of which are commercially available NAMPT activators). In some embodiments, the NAMPT activator is SBI-797812. In some embodiments, the NAMPT activator is P7C3. In some embodiments, the NAMPT activator is a compound that promotes or aids NAMPT dimerization and/or NAMPT phosphorylation. In some embodiments, the NAMPT activator is a compound that promotes or aids NAMPT dimerization. In some embodiments, the NAMPT activator is a compound that promotes or aids NAMPT phosphorylation. In some embodiments, the methods further comprise administering a second therapeutic agent selected from the group consisting of a compound useful in the treatment of diabetes, obesity, or a neurodegenerative disease. In some embodiments, the methods further comprise administering a second therapeutic agent useful in the treatment of diabetes, obesity, or a neurodegenerative disease. In some embodiments, the methods further comprise administering a second therapeutic agent useful in the treatment of obesity. In some embodiments, the methods further comprise administering a second therapeutic agent useful in the treatment of a neurodegenerative disease.

In some embodiments, the administered compound(s), or a pharmaceutically acceptable salt thereof, are a component of a pharmaceutical composition further comprising a pharmaceutically acceptable carrier.

The pharmaceutical composition my optionally comprise a neoadjuvant therapeutic agent, a chemotherapeutic agent, an immunotherapeutic agent, a lysosome inhibitor, or a calpain inhibitor, or any combination thereof.

Neoadjuvant therapeutic agents include all forms of treatment of cancer including, but not limited to, traditional chemotherapy (i.e., anti-cancer agents or chemotherapeutic agents, whether they are administered parenterally or orally), immunotherapy, small molecule enzyme or kinase inhibitors, intravesical therapies, antibody inhibitors of receptors or kinases, antibody-drug conjugates, and radiation therapy.

Examples of chemotherapeutic agents include, without limitation, methotrexate, taxol, mercaptopurine, thioguanine, hydroxyurea, cytarabine, mitomycin, cyclophosphamide, ifosfamide, nitrosourea, cisplatin, carboplatin, oxaliplatin, nedaplatin, triplatin tetranitrate, phenanthriplatin, picoplatin, satraplatin, dacarbazine, procarbizine, an etoposide, a campathecin, bleomycin, doxorubicin, idarubicin, daunorubicin, dactinomycin, distamycin A, etidium, netropsin, auristatin, amsacrine, prodigiosin, bortexomib, pibenzimol, tomaymycin, duocarmycin SA, plicamycin, mitoxantrone, asparaginase, vinblastine, vincristine, MG132, tunicamycin, oligomycin, vinorelbine, paclitaxel, docetaxel, CPT-11, gleevec, erlotinib, gefitinib, ibrutinib, crizotinib, ceritinib, flavopiridol, gemcitabine, lapatinib, navitoclax, sorafenib, regorafenib, ganetespib, irinotecan, or 5-fluorouracil, or any combination thereof. In some embodiments, the chemotherapeutic agent is a combination of agents, such as, for example, methotrexate/vincristine/doxorubicin/cisplatin (MVAC) or gemcitabine/cisplatin.

In some embodiments, the neoadjuvant agent is an immunotherapeutic agent such as, for example, nivolumab, pembrolizumab, atezolizumab, durvalab, ipilumumab, avelumab, cetuxumab, bevacizumab, or trastuzumab, or any combination thereof. In some embodiments, the neoadjuvant agent is an immunotherapeutic agent such as, for example, OPDIVO® (nivolumab), KEYTRUDA® (pembrolizumab), TECENTRIQ® (atezolizumab), IMFINZI® (durvalab), YERVOY® (ipilumumab), BAVENCIO® (avelumab), ERBITUX® (cetuxumab), AVASTIN® (bevacizumab), or HERCEPTIN® (trastuzumab), or any combination thereof.

An example of a lysosome inhibitor is chloroquine.

Examples of calpain inhibitors include, without limitation, AK275, MDL28170, PD150606, SJA6017, ABT-705253, or SNJ-1945, or any combination thereof.

In some embodiments, the ratio of the compound to the chemotherapeutic agent, the lysosome inhibitor, the immunotherapy agent, or the calpain inhibitor in the pharmaceutical compositon is from about 0.01:1 to about 100:1 w/w.

The compositions may be prepared to provide from about 0.05 mg to about 500 mg of the compound, or pharmaceutically acceptable salt thereof. The compositions may comprise from about 1 mg to about 200 mg of the compound, may comprise from about 10 mg to about 200 mg of the compound, may comprise from about 10 mg to about 100 mg of the compound, may comprise from about 50 mg to about 100 mg of the compound, may comprise from about 20 mg to about 400 mg of the compound, may comprise from about 100 mg to about 300 mg of the compound, and may comprise from about 50 mg to about 250 mg of the compound, or pharmaceutically acceptable salt thereof.

Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions.

Solid dosage forms include tablets, pills, powders, bulk powders, capsules, granules, and combinations thereof. Solid dosage forms may be prepared as compressed, chewable lozenges and tablets which may be enteric-coated, sugar coated or film-coated. Solid dosage forms may be hard or encased in soft gelatin, and granules and powders may be provided in non-effervescent or effervescent form. Solid dosage forms may be prepared for dissolution or suspension in a liquid or semi-liquid vehicle prior to administration. Solid dosage forms may be prepared for immediate release, controlled release, or any combination thereof. Controlled release includes, but is not limited to delayed release, sustained release, timed pulsatile release, and location-specific pulsatile release, and combinations thereof.

Liquid dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions may be oil-in water or water-in-oil emulsions.

In some embodiments, the oral formulation is a pill, tablet, capsule, cachet, gel-cap, pellet, powder, granule, or liquid.

The amount of compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Suitable dosage ranges for oral administration include, but are not limited to, from about 0.001 mg/kg body weight to about 200 mg/kg body weight, from about 0.01 mg/kg body weight to about 100 mg/kg body weight, from about 0.01 mg/kg body weight to about 70 mg/kg body weight, from about 0.1 mg/kg body weight to about 50 mg/kg body weight, from 0.5 mg/kg body weight to about 20 mg/kg body weight, or from about 1 mg/kg body weight to about 10 mg/kg body weight. In some embodiments, the oral dose is about 5 mg/kg body weight.

Suitable dosage ranges for intravenous administration include, but are not limited to, from about 0.01 mg/kg body weight to about 500 mg/kg body weight, from about 0.1 mg/kg body weight to about 100 mg/kg body weight, from about 1 mg/kg body weight to about 50 mg/kg body weight, or from about 10 mg/kg body weight to about 35 mg/kg body weight.

Suitable dosage ranges for other routes of administration can be calculated based on the forgoing dosages as known by one skilled in the art. For example, recommended dosages for intradermal, intramuscular, intraperitoneal, subcutaneous, epidural, sublingual, intracerebral, transdermal, or inhalation are in the range from about 0.001 mg/kg body weight to about 200 mg/kg body weight, from about 0.01 mg/kg body weight to about 100 mg/kg body weight, from about 0.1 mg/kg body weight to about 50 mg/kg body weight, or from about 1 mg/kg body weight to about 20 mg/kg body weight. Effective doses may be extrapolated from dose-response curves derived from in vitro or animal model test systems. Such animal models and systems are well known in the art.

In some embodiments, the amount of the compound administered to the mammal is from about 0.1 mg to about 500 mg.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: General Methodology

Antibody production: NAMPT Y188 phospho-specific, rabbit polyclonal antibody was raised against a C-NLDGLE (pY)KLHDFG-amide peptide SEQ ID NO: 20, which corresponds to residue Y188 of human NAMPT. The antibody was generated and affinity purified by Thermofisher, Protein Research Services, Rockford, IL.

Antibodies and chemical reagents: NAMPT/PBEF Polyclonal antibody (Catalog #11776-1-AP, Proteintech), Phospho-ALK (Tyr1604) Antibody (Catalog #3341S) and IGF-I Receptor β Antibody (catalog #3027S) from Cell Signaling Technology). ALK Monoclonal Antibody (4C5B8) (Catalog #35-4300, Thermofisher), HA-tagged peptide (Catalog #3320-205) and NAMPT activity assay kit (Catalog #CY-1251V2) from MBL International Inc. Lenti-X™ Concentrator (Catalog #631231, TAKARA BIO). ALK Monoclonal Antibody (4C5B8) (Catalog #35-4300), Halt™ Protease and Phosphatase Inhibitor (catalog #78442), Pierce™ IP Lysis Buffer (catalog #87788), Pierce™ Anti-HA Magnetic Beads (catalog #88837), Insulin Receptor Beta Polyclonal Antibody (catalog #A303-712A), Goat anti-Rabbit IgG (H+L) Secondary Antibody, HRP (catalog #31460), Goat anti-Mouse IgG (H+L) Secondary Antibody, HRP (catalog #31430), Pierce™ BCA Protein Assay Kit (catalog #23225) and PVDF Transfer Membrane, 0.2 μm, 26.5 cm×3.75 m (catalog #88520) all are purchased from Thermofisher Scientific. All other laboratory reagents were purchased from Sigma-Aldrich or Thermofisher Scientific.

Active human Recombinant Kinases: ALK Protein (Catalog: PV3867), BTK protein (catalog #PV3363), SRC protein (catalog #P3044), SYK protein (catalog #PV3857), FYN protein (catalog #P3042), FLT3 protein (catalog #PV6190), LCK protein (catalog #P3043), ABL1 protein (catalog #PV3865), EGFR protein (catalog #PV3872), ERK1 protein (catalog #PV3311) and AKT1 protein (catalog #P2999) all are purchased from Thermofisher. ALK inhibitor Ceritinib (Catalog No. S7083) is from Selleckchem (Houston, TX).

Plasmid Preparation: NAMPT-HA-WT_pLenti_MS2-P65-HSF1_GFP, NAMPT-HA-Y34F_pLenti_MS2-P65-HSF1_GFP, NAMPT-HA-Y188F_pLenti_MS2-P65-HSF1_GFP and NAMPT-HA-SS-AA_pLenti_MS2-P65-HSF1_GFP were synthesized at Genescript, Inc (Nanjing, China).

Addgene plasmids: pMSCV-mCherry-Syk (catalog #50045), Human Insulin receptor (HIR) (catalog #24049), pHAGE-ALK (catalog #116712), pHAGE-ALK-R1275Q and (catalog #116111), pHAGE-IGF1R (catalog #116752). Lenti Virus Packaging and transfection reagent: 3rd Gen. Packaging Mix & Lentifectin ComboPack (catalog #LV053-G074 from Abmgood, Canada) and PolyJet™ In Vitro DNA Transfection Reagent (catalog #SL100688) from Signagen laboratories (Frederick, MD).

Cell lines: Six ALK+ALCL cell lines (SUDHL1, SUPM2, SR786, L82, JB6 and KARPASS299), two NPM-ALK transduced human CD4+ cell lines (NAl and NA2), 2 ALK-ALCL cell lines (MAC2A, MyLa2059), four mantle cell lymphoma (MCL) cell lines (JEKO-1, MAVER, REC-1 and RL) and human embryonic kidney epithelial cell line (HEK293T) were maintained at 37° C. in RPMI 1640 (Life Technologies/ThermoFischer (Carlsbad, CA)) and DMEM supplemented with 10% fetal bovine serum and Penicillin and Streptomycin (1 mM), in a humidified atmosphere containing 5% CO₂, respectively.

EGFP and HA-tagged NAMPT-WL NAMPT-Y34F, NAMPT-Y188F and NAMPT-SS-AA HEK-HEK-293T stable cell lines: These NAMPT constructs tagged with HA and GFP in pLenti_MS2-P65-HSF1_GFP vector alone or NAMPT-WT, NAMPT-Y34F_pLenti_MS2-P65-HSF1_GFP and NAMPT-HA-Y188F_pLenti_MS2-P65-HSF1_GFP and NAMPT-SS-AA plasmids were synthesized (Genscript, Inc. (Nanjing, China).). We prepared lentivirus for these constructs using 3rd generation lentivirus packing system (Abmgood, Burlington, Canada) along with NAMPT plasmids in HEK-293T cells. The lentivirus was concentrated by using lenti-X concentrator (Takarabio, Inc. Shiga, Japan) and the constructs were transduced into NAMPT knockdown HEK293T cells by using NAMPT shRNA MISSION Lentiviral transduction particles specific for human NAMPT with puromycin selection marker with scramble or pLKo.1-puro CMV vector or NAMPT specific oligos (Sigma-Aldrich, Inc. St. Louis, MO). The stable cell lines of NAMPT-WT, Y34F, Y188F and SS-AA were selected by flow cytometry sorting of GFP positive cells. For transient expression of NAMPT and other constructs were transiently transfected in HEK293T cells using Polyjet (SignaGen Lab, Ballenger Creek, MD) above NAMPT constructs following the manufacturer's guidelines.

EGFP and HA-tagged NAMPT-WL NAMPT-Y188F and NAMPT-SS-AA (monomeric) mutant ALK+ALCL stable cell lines: To stably overexpress NAMPT-WT, NAMPT-Y188F and NAMPT-SS mutant form of human full-length constructs in SUPM2 and SUDHL1, lentivirus transduction particles were generated using HEK293T packaging cells transfected by 3rd generation lentivirus packaging kit (Abmgood, Canada). The virus particles were concentrated by using Lenti-X concentrator (Takara Bio) as per the instructions. ALK+ALCL cell lines SUPM2 and SUDHL1 cell lines were transduced with concentrated virus in the presence of polybrene and expanded for three passages; EGFP-positive cells were subjected to fluorescence-activated cell sorting (FACS) to obtain cells stably expressing NAMPT-WT, NAMPT-Y188F and NAMPT-SS-AA mutant.

Immunoblotting analysis: Proteins were extracted using cell lysis buffer (Pierce, Thermofisher) containing a cocktail of protease and phosphatase inhibitors. For western blotting, 50 g of total cell proteins were subjected to SDS-PAGE in 10 or 4-20% NuPAGE gradient gels under reducing conditions and transferred onto a nitrocellulose membrane. The blots were blocked in 5% skimmed milk in TBST and probed with primary antibodies overnight. The blots were developed using ECL western blotting detection reagent (Pierce, Thermofisher).

Immunoprecipitation and pull-down assays: HA-tagged proteins were immunoprecipitated from HEK293T or SUPM2 or SUDHL1 NAMPT-HA-EGFP-WT, NAMPT-HA-EGFP-Y188F or NAMPT-HA-GFP-SS-AA stable cell line lysates by incubation with agarose-HA antibody overnight or 3-4 hours. Beads were washed 3-4 times in lysis buffer and eluted with HA peptide and used for immunoblotting or NAMPT activity assay.

In vitro kinase assays using multiple oncogenic tyrosine kinases: Recombinant NAMPT (Abcam Inc.) was subjected to in vitro kinase assay using various recombinant active oncogenic tyrosine kinases (ALK, BTK, SYK, FYN, FLT3, LCK, SRC, ABL and EGFR) and two non-tyrosine kinases as negative control (ERK1 and AKT1) all are obtained from Thermofisher. The in vitro kinase assay was carried out by mixing 200 ng of NAMPT and with or without appropriate kinases (100 ng) in the kinase buffer (50 mM Tris.HCl, pH 7.5, 10 mM MgCl₂, 1 mM sodium fluoride, 1 mM sodium orthovanadate, 1 mM DTT and 1 mM ATP) and for 30 minutes at 30° C. Samples were heated at 95° C. for 5 minutes, separated on a 4-12% gel by SDS-PAGE and followed by western blotting using anti-NAMPT and ant-pan Tyroisne (pY100) or p-NAMPT-Y188 antibodies.

In vitro kinase assays using BTK and SYK oncogenic tyrosine kinases and phosphoproteomics analysis by mass spectrometry LC-MSMS: For this purpose, we performed large scale in vitro kinase assay as described above in triplicate. Recombinant NAMPT (Abcam Inc.) was subjected to in vitro kinase assay using BTK and SYK. Samples were heated at 95° C. for 5 minutes, separated on a 4-12% gel by SDS-PAGE and followed by western blotting using anti-NAMPT and ant-pan Tyroisne (pY100) or p-NAMPT-Y188 antibodies. The fraction of kinase assay samples was subjected to Immunoblot analysis to confirm NAMPT tyrosine phosphorylation by using p-Y100 antibody. Also, other fraction was used for NAMPT activity assay. The remaining kinase assay samples were resolved on 4-12% Native PAGE, the gel was stained with G250 and the bands were excised. Phosphotyrosine containing peptides were purified and subjected to tandem mass spectrometry for protein identification as described herein.

Blue Native polyacrylamide gel electrophoresis (Blue Native PAGE) for NAMPT dimerization study: NAMPT exist as a homodimer in order to function as an NAD biosynthetic enzyme (Revollo et al., Cell. Metab., 2007, 6, 363-375). We synthesized NAMPT gene with two key amino acids Serine 199 and Serine 200 in dimer interphase were replaced with alanine and created Alanine199 and Alaine200 (SS-AA), NAMPT-WT and NAMPT-Y188F tagged with HA and GFP in pLenti_MS2-P65-HSF1_GFP vector. The SUDHL-1 and SUP-M2 cell lines were transduced with above constructs using 3rd generation Lenti-virus packaging system and GFP positive cells were selected by flow sorting.

To investigate the expression of NAMPT-WT, NAMPT-Y188F and dimerization mutant NAMPT-SS-AA in SUPM2 and SUDHL1 cell lines, we performed western blotting and probed with p-NAMPT Y188 and HA antibodies in reducing NATIVE PAGE gel electrophoresis method.

To confirm the NAMPT dimerization and dimerization interphase mutated NAMPT-SS-AA conditions, we prepared the protein lysates in non-reducing conditions as described in Blue native PAGE (BN-PAGE) one-step isolation of protein complexes from biological membranes and total cell and tissue homogenates. It can also be used to determine native protein masses and oligomeric states and to identify physiological protein-protein interactions (Wittig et al., Nat. Protoc., 2006, 1, 418-428; and Wittig et al., Bba-Bioenergetics, 2010, 1797, 71-71). We performed Blue Native PAGE as described by others (D'Amici et al., J. Proteome Res., 2008, 7, 1326-1340). The protein samples prepared without disturbing their native conformation by using sample preparation kit as described herein. Furthermore, native polyacrylamide gel electrophoresis, can be performed using NativePAGE™ 4 to 16%, Bis-Tris, 1.0 mm, Mini Protein Gel, 10-well (Catalog #BN1002BOX), NativePAGE™ Running Buffer Kit (Catalog #BN2007), NativePAGE™ Sample Buffer (4×) (catalog #BN2003), NativePAGE™ Sample Prep Kit (Catalog #BN2008), NuPAGE® Transfer Buffer (20×) (catalog #NP0006-1) and NativeMark™ Unstained Protein Standard (catalog #LC0725) all are purchased from ThermoFisher Inc.,

NPM-ALK interaction with NAMPT requires Y188 phosphorylation and dimerization: To establish the role of NPM-ALK in NAMPT Y188 phosphorylation and protein-protein interaction, we used the ALK+ALCL cell lines SUPM2 and SUDHL-1 stable cell lines expressing vector alone, NAMPT-WT, NAMPT-Y188F and NAMPT-SS-AA (monomeric form). The cells were grown in large scale and protein lysates were prepared. The protein lysates were subjected to immunoprecipitation (IP) with anti-HA-agarose conjugated beads and kept on rotating for 2 hours in cold room. The beads were washed with lysis buffer for four times, mixed with 2×SDS-sample buffer and heated at 90° C. for 5 minutes. The samples were resolved on 10% NUPAGE gel and probed with anti-ALK, anti-p-ALK Y1604, anti-p-NAMPT Y188 and anti-HA antibodies.

NPM-ALK regulates NAMPT Y188 phosphorylation: ALK+ALCL cell lines SUDHL-1 and SUPM2 cells were treated with or without certinib for 24 hours and the cell lysates were subjected to p-NAMPT Y188 and other indicated antibodies. Human CD4+ cell lines transduced with NPM-ALK (NA1, NA2 and NA69) were treated with or without certinib for 24 hours and the cell lysates were subjected to p-NAMPT Y188 and other indicated antibodies.

In vivo NAMPT Y188 phosphorylation in HEK-293T cell lines: HEK-293 T stable cell lines with EGFP and HA-tagged NAMPT-WT and NAMPT-Y188F cells transiently transfected with catalytically active tyrosine kinases such as ALK (wild type), ALKR1275Q (point mutation variant in neuroblastoma) Insulin receptor, IGF1R, BTK and SRC family kinase SRC by using respective kinase plasmid and polyjet reagent in 10 cm culture pates. The cells were cultured for 48 hours, after that, cells were harvested and lysed in lysis buffer. The protein lysates were subjected to western blotting and probed with anti-p-NAMPT Y188, anti-HA, anti-IGF1R, anti-BTK, anti-SYK and anti-SRC antibodies. We also performed same protein lysates for immunoprecipitation (IP) with anti-HA-agarose conjugated beads as described above and probed with indicated antibodies.

NAMPT activity assay: NAMPT activity was determined using the commercial NAMPT Activity Assay Kit (Colorimetric) (Abcam #ab221819) as described by the instructions. Briefly, for in vitro kinase assay and tyrosine phosphorylated recombinant NAMPT samples were directly mixed with NAMPT activity assay buffer in 96 well plate and time course kinetic curve were created by reading optical density (O.D) at 450, was continuously monitored every 1 minute for 60 minutes using a microplate reader. The assay is based on a multi-step reaction that converts WST-1 to WST-1 formazan by NAD/NADH enzyme cycling reaction, which can be easily detected at OD 450 nm at regular intervals after the reaction is initiated to determine velocity of reaction by fitting the OD values in PRISM software to find the slope of reaction and finally calculated the relative NAMPT enzyme activity when compared to control or drug treated or NAMPT-WT vs. NAMPT-Y188F or NAMPT-SS-AA purified proteins. The relative NAMPT activities were calculated by normalization to the total protein abundance of the extracts in triplicate.

NAMPT phospho-tyrosine motif derived peptides inhibit NAMPT activity: Among NAMPT tyrosine phosphorylation site identified by mass spectrometry, we selected six phospho motif regions and synthesized 11-14 amino acid peptides (Genescript Inc.). The peptides are: Pep-i: PNTSKVYSYFECR (SEQ ID NO: 14); Pep-2: KLRKVKYEETVF (SEQ ID NO: 15); Pep-3: GWNYILEKYDGHL, (SEQ ID NO: 16); Pep-4: KLHDFGYRGVSSQE (SEQ ID NO: 1); and Pep-5: KGDLEEYGQDL (SEQ ID NO: 17). The above peptides were dissolved in Tris-HCl buffer pH 7.4 at 1.0 mM concentration and used in the NAMPT activity assay at 10 μM to 200 μM final concentration in the reaction volume.

NMN and NAD metabolomics analysis: SUPM2 cells were treated with DMSO or certinib (50 nM) for 15 hours in complete RPMI media containing 10% FBS, 2 mM Glutamine and 1% Pen/Strep antibiotics. Cell pellets were snap frozen in liquid nitrogen and processed for LC-MS/MS analysis as described.

Cellproliferation and colonyformation assay: ALK+ALCL cell line, SUDHL1 cells expressing NAMPT-HA-EGFP-WT, NAMPT-HA-EGFP-Y188F and NAMPT-HA-EGFP-SS-AA mutant cells were plated at a concentration of 1×10⁴ cells/well in 96 well plates. Cells were grown 72 hours. Cell proliferation was assessed by measuring EGFP fluorescence excitation peak at 488 nm and an emission peak at 530 nm in plate reader. Colony formation assay was performed with MethoCult methylcellulose-based media as per manufacturer's protocol (Stemcell Technologies, Vancouver, British Columbia, Canada). After 14 days, colonies were imaged under iBright 1500 and colony density was measure using IMAGEJ software. Statistical analysis: Statistical analysis and graphical presentation was performed using GraphPad Prism 4.0.

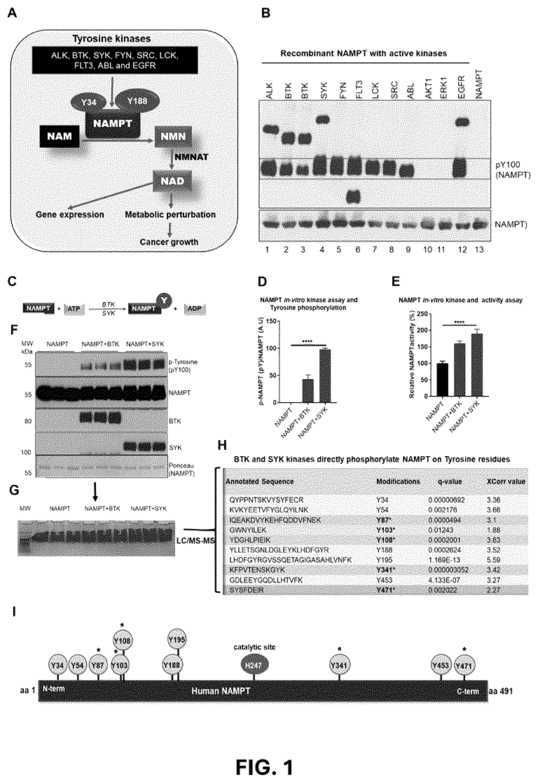

It is our position that, due to the experiments described herein, oncogenic tyrosine kinases directly phosphorylate NAMPT and its increased enzyme activity drives tumor metabolism in hematological malignancy (FIG. 1, panel A).

### Example 2: In Vitro Kinase Assay Analysis Reveals Tyrosine Kinases Directly Phosphorylating NAMPT on Tyrosine Residues

To evaluate whether tyrosine phosphorylation of NAMPT occurs directly by various oncogenic kinases, we carried out in vitro kinase assays using commercially available recombinant human NAMPT protein and catalytically active human recombinant tyrosine kinases, anaplastic lymphoma kinase (ALK), bruton tyrosine kinase (BTK), spleen tyrosine kinase (SYK), SRC family tyrosine kinases, SRC, Proto-oncogene tyrosine-protein kinase, SRC, FYN, LCK proto-oncogene, fins-like tyrosine kinase 3 (FLT3), ABL proto-oncogene 1, epidermal growth factor receptor (EGFR) and two non-tyrosine kinases extracellular signal-regulated kinase 1 (ERK1) and AKT serine/threonine kinase as negative controls. NAMPT alone with all the in-vitro kinase assay buffer accept active tyrosine kinase served as a control. NAMPT and tyrosine kinases in the presence of kinase buffer were incubated at 37° C. for 30 minutes and the reaction was terminated by adding 2×SDS-sample buffers and heated at 90° C. for 5 minutes. The samples were subjected to Immunoblotting and probed with NAMPT and pan-tyrosine antibody (pY100). The results revealed that NAMPT is tyrosine phosphorylated in the presence of active tyrosine kinases but not in negative control or ERK1/AKT1 kinase (FIG. 1, Panel B).

### Example 3: Phosphoproteomic Analysis Reveals Tyrosine Phosphorylation of NAMPT on Multiple Tyrosine Residues

The in vitro kinase assay results revealed that NAMPT is tyrosine phosphorylated by various oncogenic tyrosine kinases but we focused our research on BTK, SYK and ALK mediated signaling because of their role in mantle cell lymphoma and ALK+ALCL pathogenies. Next, the inventors focused on BTK, SYK, and ALK-mediated signaling to identify the NAMPT tyrosine phosphorylated sites by scaling up an in-vitro kinase assay and liquid chromatography-mass spectrometry (LC-MS/MS) based phosphoproteomics approach. To this end, in triplicate, in vitro kinase assays were performed using SYK and BTK kinase as shown in the schematic (FIG. 1C). The fraction of in-vitro translated samples were immunoblotted and probed with pan-tyrosine (pY100), NAMPT, SKY, and BTK. The results revealed tyrosine phosphorylation of NAMPT (FIG. 1D). The densitometry values showed a higher level of NAMPT phosphorylation in the presence of SYK kinase compared to BTK (Fig. E). Similarly, a fraction of the in-vitro kinase samples were used for the NAMPT activity assay. The results revealed that tyrosine phosphorylation of NAMPT increased the enzyme activity almost two-fold (FIG. 1F). Next, the remaining in-vitro kinase samples were resolved on SDS-PAGE and subjected to LC/MS/MS and phosphoproteomics analysis (FIG. 1G). The results revealed NAMPT phosphorylation on ten Tyrosine (Y) residues, Y34, Y54, Y87*, Y103*, Y108*, Y188, Y195, Y341*, Y453 and Y471*as shown in the table (FIG. 1H). The inventors demonstrated for the first time five tyrosine residue phosphosphorylation sites of NAMPT ever reported in the databases as shown with bold and asterisk. Our phosphoproteomics data identified ten tyrosine phosphorylated residues, illustrated in the schematic representation of human NAMPT phosphorylation sites (FIG. 1I).

### Example 4: Tyrosine Phosphorylation Domains Regulate NAMPT Enzyme Activity

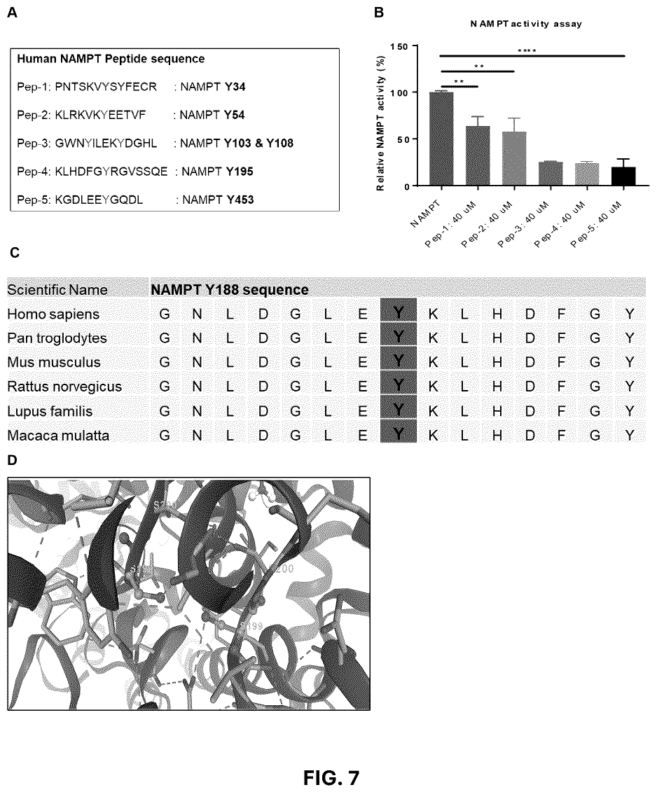

Based on the NAMPT tyrosine phosphorylation site, the inventors further synthesized NAMPT constructs with Y-F mutation with a lentivirus-carrying vector, which included the variants NAMPT-Y34F, NAMPT-Y54F, NAMPT-Y103F, NAMPT-Y188F, NAMPT-Y195F, and NAMPT-Y453F at GenScript. These variants were further expressed in HEK293T cells and evaluated through NAMPT activity assays, and the inventors observed that NAMPT Y188F residue showed a significant decrease in NAMPT activity (data not shown). Next, to investigate the role of tyrosine phosphorylation of NAMPT residues and their surrounding amino acids (10-13 amino acid peptides), which were referred to as phospho-domains, the inventors selected six tyrosine residue domains and 10-14 amino acid peptides from the ten identified residues. These peptides were synthesized at Genscript, Inc. The selected sequences and corresponding tyrosine domains of NAMPT peptides are, Pep-1: PNTSKVYSYFECR (NAMPT Y34) (SEQ ID NO: 14), Pep-2: KLRKVKYEETVF (NAMPT Y54) (SEQ ID NO: 15), Pep-3: GWNYILEKYDGHL (NAMPT Y103 & Y108) SEQ ID NO: 16), Pep-4: KLHDFGYRGVSSQE (NAMPT Y195) (SEQ ID NO: 1) and Pep-5: KGDLEEYGQDL (NAMPT Y453) (SEQ ID NO: 17). To evaluate the inhibitory effect of the self-derived NAMPT peptides 1-5 as shown (FIG. 7A) on NAMPT activity, we pre-incubated recombinant NAMPT protein with the peptides at 40 uM concentrations for 10-15 minutes and then assessed the NAMPT activity. The results indicated that all selected phospho-domains inhibited NAMPT activity, with Pep-3 to Pep-5 causing a significant reduction of more than 50% in enzyme activity (FIG. 7B). Based on these results, the inventors chose to focus their research more closely on two specific tyrosine residues, Y188 and Y195. The NAMPT Y188 is highly conserved among many species (FIG. 7C). This decision was reasoned based on experimental data generated together with their proximity to the NAMPT dimerization residues Ser199 and Ser200 (FIG. 7D), as well as the inventors findings related to the inhibition of NAMPT activity mediated by Pep-4 (KLHDFGYRGVSSQE, SEQ ID NO: 1) and earlier report of NAMPT Y188 phosphorylation in ALK+ALCL cell lines (Boccalatte, F. E. et al. Blood113, 2776-2790, doi:10.1182/blood-2008-06-161018 (2009).

### Example 5: Tyrosine Phosphorylation of NAMPT in ALK+ALCL Lymphomas

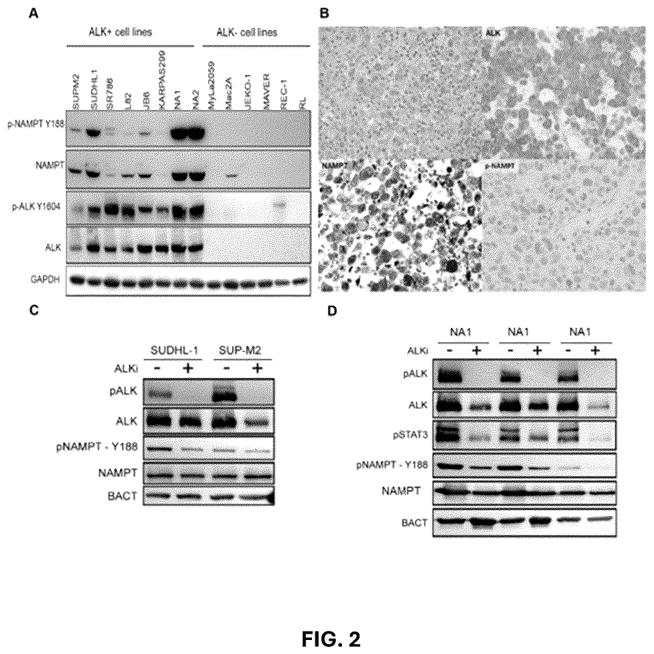

To validate the phosphorylation of NAMPT Y188 in a cellular system, the inventors custom-developed a novel antibody by injecting the corresponding peptide C-NLDGLE(pY)KLHDFG-amide (SEQ ID NO: 20) into two rabbits. This process was conducted in collaboration with ThermoFisher, Inc., followed by affinity purification. The phospho-NAMPT Y188 antibody was validated and tested on ALK+ALCL and MCL cell lines. The inventors' observations showed robust expression of NAMPT and confirmed that tyrosine phosphorylation occurred only in the ALK+ALCL cell lines. In contrast, no phosphorylation was detected in the MCL cell lines (FIG. 2A). Next, the NAMPT Y188 phosphorylation in ALK+ALCL patient-derived biopsy samples was validated by performing IHC. The results revealed NAMPT Y188 phosphorylation (FIG. 2B). To assess the ALK-mediated NAMPT phosphorylation further, the inventors treated ALCL cell lines with the small molecule ALK inhibitor, ceritinib. The results revealed that inhibition of ALK phosphorylation also inhibited NAMPT Y188 phosphorylation (FIG. 2C). The inventors also tested these findings in CD4 cells transduced with NMP-ALK (NA1), and the results revealed corroborative findings (FIG. 2D).

### Example 6: Expression of Phosphorylated NAMPT Y188 in NPM-ALK Positive ALCL Patient Biopsy Samples

To establish that NAMPT protein, both total and its Y188-phosphorylated form, is expressed not only in ALK+ TCL-derived cell lines but also in primary ALK+ TCL cells, we examined formalin-fixed, paraffin-embedded diagnostic tissue samples from eight cases of ALK+ TCL by immunohistochemistry (FIG. 2, Panel B; a representative image). As can be seen, the malignant ALK+ TCL cells identified by morphology (upper left panel) and expression of ALK (upper right) strongly expressed NAMPT (lower right) while, non-malignant lymphocytes did not express this protein at a detectable concentration. Expression of phospho-NAMPT Y188 was also clearly detectable in the malignant ALK+ TCL cells (lower right panel); the typically less robust intensity of the staining reflects technical limitations of the antibody in this immunohistochemical assay with the formalin fixed tissues. Despite this technical limitation, these findings indicate that NAMPT expression in both total and Y188-phosphorylated form is essentially universal in ALK+ TCL cells.

### Example 7: Oncogenic Mutation on ALK Enhances NAMPT Y188 Phosphorylation and Enzymatic Activity

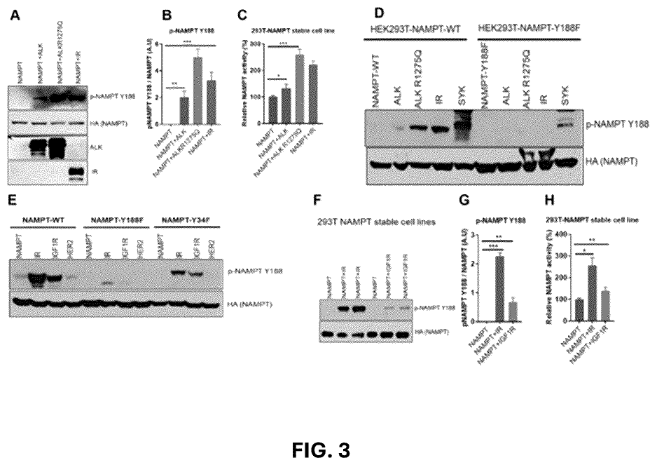

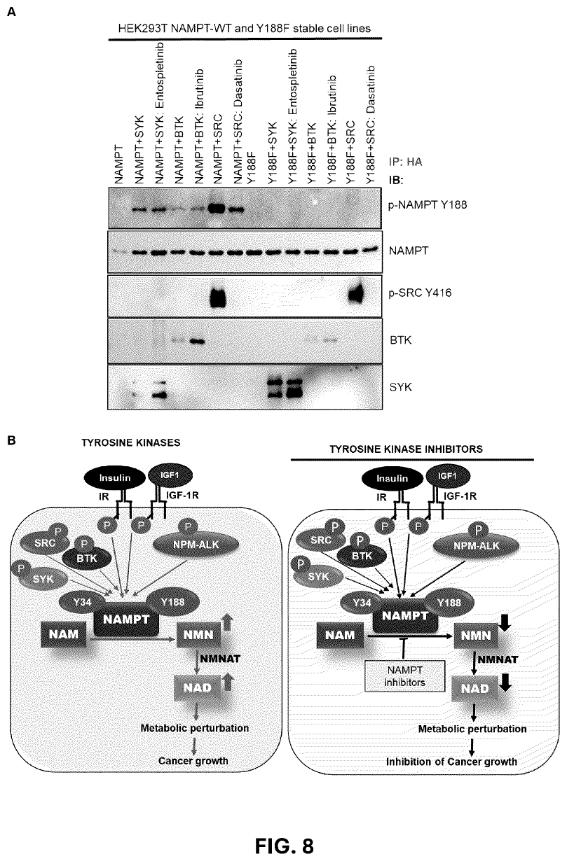

To investigate the effect of oncogenic mutation on ALK and its role in the phosphorylation of NAMPT at Y188, the inventors performed an in-vitro kinase assay on recombinant NAMPT in the presence of active ALK wild type, ALK R1275Q mutant, which is implicated in sporadic and familial neuroblastoma (Montavon, G. et al. Oncotarget 5, 4452-4466, doi:10.18632/oncotarget.2036 (2014)) and human insulin receptor (IR). Since ALK belongs to the insulin receptor family, the inventors reasoned that Insulin receptors phosphorylate NAMPT. The samples were subjected to immunoblotting and probed with antibodies targeting p-NAMPT Y188, NAMPT, ALK, and IR. The results indicated that NAMPT was significantly phosphorylated in the presence of mutated ALKR1275Q and IR compared to wild-type ALK. This phosphorylation was not observed when NAMPT was used alone (FIG. 3A). The densitometry values showed a higher level of NAMPT phosphorylation in the presence of ALKR1275Q and IR compared to ALK (FIG. 3B). Similarly, the fraction of samples was subjected to NAMPT activity assay. The results revealed that a modest increase in wild-type ALK (45%), the highest rise in ALKR1275Q point mutation (275%), and IR (220%) conditions (FIG. 3C). To investigate NAMPT phosphorylation by tyrosine kinases in cellular context, the inventors synthesized NAMPT wild type gene tagged with HA and GFP in pLenti_MS2-P65-HSF1_GFP vector alone or NAMPT-HA-Y34F_pLenti_MS2-P65-HSF1_GFP and NAMPT-HA-Y188F_pLenti_MS2-P65-HSF1_GFP constructs (Genscript, Inc.). The NAMPT-HA-WT, NAMPT-HA-Y34F, and NAMPT-HA-Y188F constructs were transduced into NAMPT knockdown HEK293T. The stable cell lines were transiently expressed with ALK wild type, ALKR1275Q, the human insulin receptor (IR), and SYK plasmids. The cell lysates were prepared after 48-hour transient transfection of the kinases, and protein samples were subjected to western blotting. The results showed that NAMPT-WT cells transfected with ALKR1275Q and IR exhibited the highest levels of NAMPT tyrosine phosphorylation. In contrast, the ALK wild type showed less phosphorylation, and no phosphorylation was observed in the NAMPT Y188F condition (FIG. 3D). These results agree with in-vitro kinase assay data. The inventors conducted similar experiments by transfecting constructs for IR, IGF1R, and HER2 into stable cell lines: NAMPT-WT, NAMPT-Y188F, and NAMPT-Y34F. The results indicated that NAMPT Y188 phosphorylation was nearly undetectable in the Y188F condition. However, in the Y34F condition, the inventors observed a reduction in NAMPT Y188 phosphorylation (FIG. 3E). Next, the inventors transfected NAMPT-WT stable cell lines with IR and IGF1R constructs. They tested NAMPT Y188 phosphorylation and activity assay. The results indicated that NAMPT was significantly phosphorylated in the presence of IR compared to IGF1R (FIG. 3F). The densitometry values showed a higher level of NAMPT phosphorylation in the presence of IR and compared to IGF1R (FIG. 3G). The protein lysates were immunoprecipitated (IP) with anti-HA conjugated magnetic beads and eluted NAMPT-HA proteins with HA peptide. These eluted proteins were used for the NAMPT assay. The results revealed IR transfected cells with the highest 275% and IGF1R (135%) activity (FIG. 3H). To further investigate, the inventors also tested the tyrosine phosphorylation of NAMPT by other kinases, specifically SYK, BTK, and SRC, in HEK293T cells. The results revealed the phosphorylation of NAMPT at Y188 (FIG. 8A). A schematic illustrating NAMPT Y188 phosphorylation and the regulation by various tyrosine kinases (TK) and tyrosine kinase inhibitors (TKI) is shown (FIG. 8B).

### Example 8: Human Insulin Receptor Directly Phosphorylates NAMPT on Tyrosine 188 Residue in In Vitro Kinase Assay

Insulin and Insulin growth factor-1 (IGF-1) control a wide variety of biological processes by activation on two closely related Insulin receptor (IR) and IGF-1R tyrosine kinase receptors and initiates a cascade of phosphorylation events that control many aspects of metabolism and growth (Boucher et al., Csh Perspect Biol., 2014, 6). The IGF-1R inhibitor GSK1838705 showed potent inhibition of ALK and tumor growth in NPM-ALK+ cell line derived xenograft models (Sabbatini et al., Mol. Cancer, 2009, Ther., 8, 2811-2820). To interrogate phosphorylation of NAMPT at Y188 by IR, we performed in vitro kinase assays in the presence of recombinant IR and NAMPT protein. The samples were immunoblotted and probed with p-NAMPT Y188, NAMPT and IR. The results revealed NAMPT tyrosine phosphorylation only in the presence of active IR but not in NAMPT alone (FIG. 3, Panel D). These results led us to hypothesize that NAMPT is a substrate of ALK and Insulin receptor tyrosine kinase.

### Example 9: The Dimerization of NAMPT is Critical for Tyrosine Phosphorylation and NPM-ALK Interaction

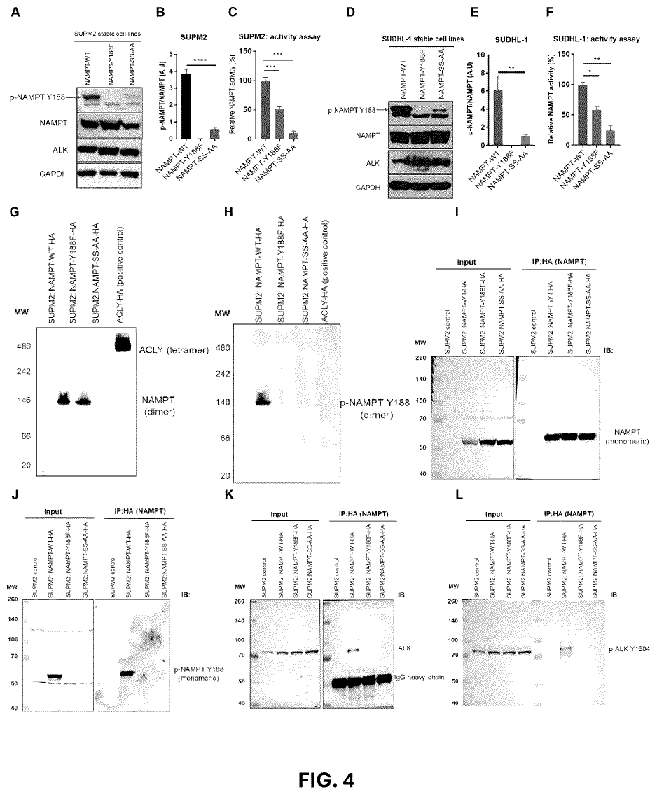

NAMPT exists as a homodimer to function as a NAD biosynthetic enzyme (Revollo, J. R. et al. Cell Metab 6, 363-375, doi:10.1016/j.cmet.2007.09.003 (2007)). The inventors synthesized the NAMPT gene with Serine199 and Serine 200 to Alanine mutation (SS-AA), NAMPT-WT, and NAMPT-Y188F tagged with HA and GFP in pLenti_MS2-P65-HSF1GFP vector. Having established the role of ALK and Insulin receptors in NAMPT phosphorylation, the inventors focused on the ALK+ALCL disease model for further biochemical and biological significance. Thus two ALK+ALCL SUPM2 and SUDHL1 stable cell lines were established with NAMPT-WT, NAMPT-Y188F, and NAMPT-SS-AA gene tagged with HA and GFP in pLenti_MS2-P65-HSF1 GFP vector. To investigate the expression of NAMPT-WT, NAMPT-Y188F, and dimerization mutant NAMPT-SS-AA in SUPM2 and SUDHL1 cell lines, the inventors performed western blotting and probed with p-NAMPT Y188 and HA (NAMPT) antibodies. The results revealed that SUPM2 cell line results showed a complete lack of phosphorylation in NAMPT Y188F expressing cell lines and significant loss of NAMPT Y188 phosphorylation in NAMPT-SS-AA mutant expressing cell lines in comparison to NAMPT-WT cell lines (FIG. 4A). Next, densitometry quantitation on the level of p-NAMPT Y188 vs. NAMPT level was measured based on three independent blots. The inventors observed a complete loss of p-NAMPT Y188 signal in Y188F expressing cells and a significant decrease of p-NAMPT Y188 signal in SS-AA (5-fold; p-value; 0.003) in comparison to NAMPT-WT cells (FIG. 4B). Similarly, they also evaluated the effect of NAMPT Y188F and non-dimerized NAMPT-SS-AA on enzyme activity. The results revealed 50% and 90% loss of NAMPT activity in NAMPT-Y188F and NAMPT-SS-AA proteins, respectively (FIG. 4C). To reproduce these results in other ALK+ALCL cell lines, the same experiments were performed with SUDHL1 cell lines and similar results were observed. NAMPT phosphorylation level (FIG. 4D), p-NAMPT Y188 densitometry quantitation (FIG. 4E), and NAMPT activity assay (FIG. 4F) the results are corroborative with the SUPM2 cell line. Next, to assess the impact of tyrosine phosphorylation of NAMPT-WT, Y188F, and SS-AA forms on dimerization, the protein lysates were separated on native-PAGE electrophoresis without adding any reducing agents to keep the NAMPT dimer intact and performed immunoblotting with p-NAMPT Y188, HA (NAMPT) antibodies. The results indicated that NAMPT-WT and Y188F formed a dimer, whereas the SS-AA mutant did not (FIG. 4G). The inventors also demonstrated that ACLY is a known tetramer that serves as a positive control. Similarly, the results showed that NAMPT-WT is tyrosine phosphorylated but not Y188F mutant (FIG. 4H). The interaction of NPM-ALK with NAMPT was explored. To examine the effects of NAMPT-WT, NAMPT Y188F, and the dimer mutant on the protein-protein interaction with NPM-ALK, immunoprecipitation (IP) using an HA antibody was performed. The inventors then performed standard SDS-PAGE in two identical gels and probed the results with p-NAMPT Y188, HA antibodies. The findings 25 showed that all three forms of HA-tagged NAMPT-NAMPT, NAMPT-WT, and the SS-AA variant were detectable in both the input samples and the IP samples when using the HA antibody (FIG. 4I). Next, the identical blot was probed with p-NAMPT Y1604 and results revealed that phosphorylation of wild type NAMPT (FIG. 4J). The same blots were then stripped and probed for ALK and p-ALK Y1604. The results indicated that the interaction between NAMPT and NPM-ALK and p-ALK occurred only in the wild-type form of NAMPT, but not in the Y188F or SS-AA mutant forms (FIG. 4 K, L).

### Example 10: ALK Point Mutation Increases NAMPT Tyrosine 188 Phosphorylation

Next we further investigated, the role of ALK wild type and whether point mutations in full-length ALK also regulate NAMPT Y188 phosphorylation, we transiently transfected pHAGE-ALK (Addgene, plasmid #116712) and ALK R1275Q mutant, pHAGE-ALK-R1275Q (Addgene, plasmid #116111) and IR plasmid and pMSCV-mCherry-Syk (Addgene, Plasmid #50045) into NAMPT-WT and Y188F HEK293T stable cell lines. The cell lysates were prepared after 48-hour transient transfection of the kinases and protein samples were subjected to western blotting. Western blotting of the proteins using an anti-phospho-NAMPT Y188 antibody revealed that highest level of NAMPT tyrosine phosphorylation observed in NAMPT-WT cells transfected with ALK R1275Q and IR and no phosphorylation in NAMPT-Y188F mutant cell lines. These results suggest that point mutations in full-length ALK increase NAMPT tyrosine phosphorylation.

### Example 11: NAMPT Tyrosine Phosphorylation Activates Enzyme Activity

As we established the role of oncogenic kinases as well as physiologically important receptor kinase, Insulin receptor on NAMPT phosphorylation, next we asked its critical role in activity regulation. To investigate, we used the same approach as mentioned above by transiently expressing ALK, ALKR1275Q and IR kinases into NAMPT-WT stable cell lines. To confirm the phosphorylation ofNAMPT, we assessed the level of phosphorylated NAMPT in cell lysates by Immunoblotting (FIG. 4, Panel D) and phosphorylation was quantified by densitometry by using three independent experiments. The results revealed that ALKR12&5Q and IR has highest level of NAMPT phosphorylation as compared to ALK wild type. Next, to investigate the role NAMPT phosphorylation on its enzyme activity, cell lysates were subjected to immunoprecipitation with HA agarose conjugated beads and eluted NAMPT-HA proteins with HA peptide. These eluted proteins were used for NAMPT assay. The results revealed that modest increase in wild type ALK (45%), highest increase in ALKR1275Q point mutation (275%) and IR (220%) transfected 293T cells.

### Example 12: Insulin Receptor Enhances NAMPT Tyrosine Phosphorylation and Activity

To assess the direct evidence of Insulin receptor mediated tyrosine phosphorylation of NAMPT, we transiently expressed IR in HEK293T-NAMPT-WT and NAMPT-Y188F stable cell lines. The protein lysates were confirmed for the NAMPT phosphorylation by western blotting with p-NAMPT Y188. The results revealed NAMPT phosphorylation in IR transfected with NAMPT-WT cells but not in NAMPT-Y188F cells. Next to investigate enzyme activity, we purified HA-tagged NAMPT-WT protein from the same lysates by immunoprecipitation with HA agarose conjugated beads and eluted NAMPT-HA proteins with HA peptide. The eluted enzyme used in NAMPT activity assay. These results revealed Insulin receptor activated 260% increased NAMPT activity on NAMPT-WT but not in NAMPT-Y188F protein. These results suggest a strong correlation between phosphorylation and activation of NAMPT.

### Example 13: Computational Modeling and Molecular Docking Reveal ALK K1150 and NAMPT Y188 Motif Interaction

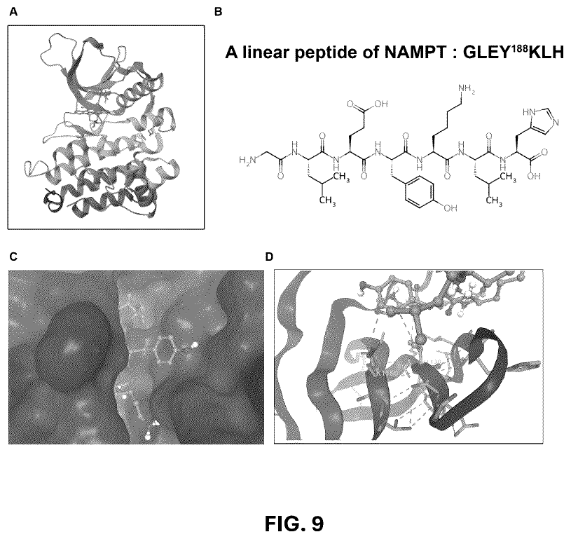

The inventors hypothesized that NPM-ALK-mediated NAMPT phosphorylation might involve the same motif responsible for protein-protein interactions. To explore this hypothesis, they employed computational and molecular docking approaches for small peptides using freely available web-based tools as described (Eberhardt, J., et al. AutoDock Vina 1.2.0: J Chem Inf Model 61, 3891-3898, doi:10.1021/acs.jcim.1c00203 (2021) and Rentzsch, R. et al. Briefings in Bioinformatics 16, 1045-1056, doi:10.1093/bib/bbv008 (2015)). The crystal structure of the Anaplastic Lymphoma Kinase Catalytic Domain is shown (FIG. 9A). We docked seven amino acids, a linear peptide of NAMPT, placing the NAMPT Y188 residue in the center (GLEY¹⁸⁸KLH) as shown (FIG. 9B) to the existing PDB structure of human ALK (PDB, 3LCS) to investigate potential interactions between NAMPT and NPM-ALK via the NAMPT Y188 domain. The results indicated that the NAMPT residue in the seven-amino-acid peptide forms hydrophobic bonds with Valine 1130 (V1130) and lysine 1150 (K1150) (Hallberg et al. Ann Oncol 27 Suppl 3, iii4-iii15, doi:10.1093/annonc/mdw301 (2016)) significant residues located in the ATP-binding region of the ALK protein, which is essential for ALK's enzymatic activity (FIGS. 9C & 9D). The NAMPT Y188 residue also forms hydrophobic bonds with L1195 and L1256 amino acids.

### Example 14: Dimerization of NAMPT is Critical for NAMPT Tyrosine Phosphorylation

NAMPT exist as a homodimer in order to function as an NAD biosynthetic enzyme. We synthesized NAMPT gene with Serine199 and Serine 200 to Alanine mutation (SS-AA), NAMPT-WT and NAMPT-Y188F tagged with HA and GFP in pLenti_MS2-P65-HSF1_GFP vector. Since, we established the role of ALK and Insulin receptor in NAMPT phosphorylation, we focused ALK+ALCL disease model for further biochemical and biological significance. Thus, we established two ALK+ALCL SUPM2 and SUDHL1 stable cell lines with NAMPT-WT, NAMPT-Y188F and NAMPT-SS-AA gene tagged with HA and GFP in pLenti_MS2-P65-HSF1_GFP vector. The cells were transduced with above constructs using 3rd generation Lenti-virus packaging system and GFP positive cells were selected by flow sorting.

To investigate the expression of NAMPT-WT, NAMPT-Y188F and dimerization mutant NAMPT-SS-AA in SUPM2 and SUDHL1 cell lines, we performed western blotting and probed with p-NAMPT Y188 and HA antibodies. SUPM2 cell line results revealed a complete lack of phosphorylation in NAMPT Y188F expressing cell lines and significant loss of NAMPT Y188 phosphorylation in NAMPT-SS-AA mutant expressing cell lines in comparison to NAMPT-WT cell lines (FIG. 4, Panel A). Next, we measured densitometry quantitation on the level of p-NAMPT Y188 vs. NAMPT level based on three independent blots. We observed complete loss of p-NAMPT Y188 signal in Y188F expressing cells and significant decrease of p-NAMPT Y188 signal in SS-AA (5-fold p-value; 0.003) in comparison to NAMPT-WT cells (FIG. 4, Panel B). Similarly, we also evaluated the effect of NAMPT Y188F and non-dimerized NAMPT-SS-AA on enzyme activity. The results revealed, 50% and 90% loss of NAMPT activity in NAMPT-Y188F and NAMPT-SS-AA proteins respectively 15 (FIG. 4, Panel C). To reproduce these results in other ALK+ALCL cell line, we performed same experiments with SUDHL1 cell lines and observed similar results. NAMPT phosphorylation level (FIG. 4, Panel D), p-NAMPT Y188 densitometry quantitation (FIG. 4, Panel E) and NAMPT activity assay (FIG. 4, Panel F) the results are in corroborative with SUPM2 cell line.

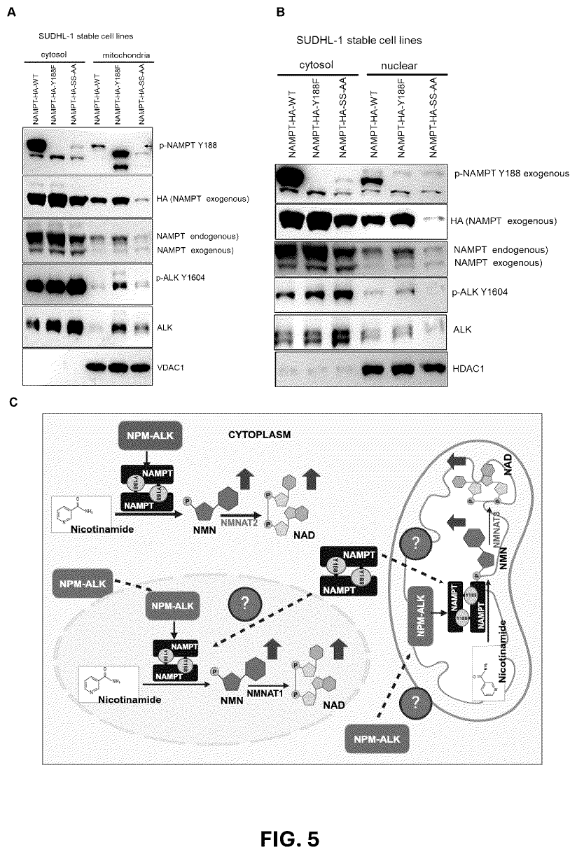

The inventors demonstrated that many oncogenic kinases directly phosphorylate NAMPT, and they observed for the first time that NPM-ALK also exists in the mitochondria of ALK+ALCL cells. A previous study by others also showed that several oncogenic kinases such as c-Abl, ErbB2, SRC, and FGFR1 translocation into mitochondria (Ding, Y. et al. Nat Commun 3, 1271, doi:10.1038/ncomms2236 (2012); Djeungoue-Petga, M. A. et al. Cell Death Dis 10, 940, doi:10.1038/s41419-019-2134-8 (2019); Hitosugi, T. et al. Mol Cell 44, 864-877, 25 doi:10.1016/j.molcel.2011.10.015 (2011); and Kumar, S. et al. J Biol Chem 276, 17281-17285, doi:10.1074/jbc.M101414200 (2001)). Despite prior investigations by others, NAMPT's role and localization to mitochondrial and nuclear compartments remained unclear. To investigate the role of NAMPT tyrosine phosphorylation and its translocation into other compartments, such as mitochondria, the inventors used commercially available kits to fractionate cytosolic/mitochondrial and cytosolic/nuclear proteins from SUDHL-1 cells that stably express NAMPT-WT, Y188F, and SS-AA cells. The protein lysates were separated on SDS-PAGE and blotted for indicated antibodies. The results revealed that NAMPT is translocated into mitochondria (FIG. 5A). The translocation of NAMPT is reduced in monomeric form NAMPT, and it shows that dimerization of NAMPT is required for mitochondrial translocation. Next, based on their understanding that certain nuclear proteins rely on NAD for their function and that NAMPT's translocation to the nucleus potentially regulates NAD synthesis the inventors fractionated the ALK+ALCL cell line SUPM2 into cytosolic and nuclear fractions, and subjected the lysates to immunoblotting with the indicated antibodies. The results showed an increased dimeric form of NAMPT nuclear translocation (FIG. 5B). The schematic illustrates the translocation of NAMPT across various compartments within the cells (FIG. 5C).

### Example 15: NPM-ALK Interaction with NAMPT Required NAMPT Dimerization

Since, we established ALK directly phosphorylate NAMPT on Y188 residue and it is highly phosphorylated in NPM-ALK positive ALCL cell lines, we reasoned that NPM-ALK also interacts with NAMPT in cellular system. To investigate this, we utilized NAMPT-WT, NAMPT-Y188F and NAMPT-SS-AA constructs expressing SUPM2 and SUDHL-1 stable cell lines and performed immunoprecipitation (IP) with anti-HA agarose conjugated beads as described in method section.

The input (left panel) as well as IP (right panel) samples were resolved on 10% NUPAGE gels and probed with anti-p-ALK Y1604 and ALK antibodies. The results revealed that NPM-ALK (phosphorylated and non-phosphorylated form) detected by ALK and p-ALK Y1604 antibodies in input samples (left panel) in all conditions but the NPM-ALK interaction with NAMPT occurred only in NAMPT wild type condition but not in phosphorylation defective or phospho-mutant NAMPT-Y188F and NAMPT-SS-AA monomeric form condition (FIG. 6, Panel A and Panel B). Next, we probed the same blots with anti-HA (for NAMPT) and p-NAMPT Y188 antibodies. The results revealed that expression of NAMPT (HA) in input as well as IP samples were equal in all three conditions (FIG. 6, Panel C) but the NAMPT Y188 phosphorylation occurred only in the presence of NAMPT wild type condition but not in phosphorylation defective or phospho-mutant NAMPT-Y188F and NAMPT-SS-AA monomeric form condition (FIG. 6, Panel D). Since we showed, NAMPT Y188F, non-phosphorylating form of NAMPT and NAMPT-SS-AA monomeric form disrupted the NPM-ALK interaction, next we validated the role of NAMPT-SS-AA on dimerization of NAMPT by performing Blue Native polyacrylamide gel electrophoresis (Blue Native PAGE). The cells from SUPM2 cell lines expressing NAMPT-WT, NAMPT-Y188F, NAMPT-SS-AA and as a positive control known tetrameric form of human ACLY-HA expressing HEK293T cells were lysed in native gel electrophoresis lysis buffer as described in method section. The samples resolved on NATIVE PAGE gel and probed with anti-HA and p-NAMPT Y188 antibodies. The results revealed thatNAMPT-SS-AA mutation form disrupted NAMPT dimerization (FIG. 6, Panel E) and p-NAMPT Y188 phosphorylation (FIG. 6, Panel F). To further these results confirmed NAMPT Y188 phosphorylation occurred only in NAMPT wild type condition.

### Example 16: SYK, BTK and SRC Kinases Interacts with NAMPT and Phosphorylate NAMPT on Y188 Residue

Based on the in-vitro kinase using SYK, SRC and BTK kinases on NAMPT tyrosine phosphorylation, we further investigated the role of SYK, SRC and BTK kinases in-vivo conditions on NAMPT Y188 phosphorylation by transfecting these kinases in HEK293T cells stably expressing NAMPT-WT-HA and NAMPT-Y188F-HA constructs as described in method section. The cells with each condition were treated with or without appropriate tyrosine kinase inhibitors such as SYK with entospletinib (1.0 uM), BTK with ibrutinib (1.0 μM) and SRC with dasatinib (1.0 μM) for overnight. The lysates were prepared and subjected to IP with anti-HA agarose conjugated beads and resolved on 10% NuPAGE gels. The blots were probed with p-NAMPT Y188, HA, SYK, BTK and p-SRC antibodies. The results revealed that SYK and BTK mediated phosphorylation on NAMPT Y188 is not changed in the presence of SYK and BTK inhibitor's—however there is a significant difference in SRC kinase inhibitor dasatinib treated samples. Similarly, we probed the same blot with HA (NAMPT), SYK, BTK and p-SRC. The results revealed that, SYK, BTK and SRC kinases interacts with NAMPT wild type as well as NAMPT-Y188F, phosphodefective form.

### Example 17: NAMPT Tyrosine Phosphorylation Regulates NMN and NAD Metabolism in Cancer

Having established that phosphorylation of NAMPT Y188 regulates its enzymatic activity, we sought to evaluate its impact on NMN and NAD metabolism as shown in schematic (FIG. 8, Panel A). We performed high-performance liquid chromatography-tandem mass spectrometry (LC-MS/MS) method as described (Revollo et al., J. Biol. Chem., 2004, 279, 50754-50763) on SUPM2 ALK+ALCL cells treated with DMSO or ALK small molecule inhibitor certinib (50 nM) for 15 hours. ALK inhibition led to significant decrease in NMN a direct product of NAMPT and its end product NAD+. We also used NMN and NAD standards in our assay to confirm the authenticity of the NMN and NAD metabolites in our study.

### Example 18: NAMPT Tyrosine Phosphorylation Regulates NMN and NAD Metabolism in Cancer

After confirming that the phosphorylation of NAMPT Y188 regulates its enzymatic activity, the inventors aimed to assess its effects on NMN and NAD metabolism, as illustrated in the schematic (FIG. 6A). They employed a high-performance liquid chromatography-tandem mass spectrometry (LC-MS/MS) method, as previously described, to analyze SUPM2 ALK+ALCL cells treated with DMSO or the ALK small molecule inhibitor certinib (50 nM) for 15 hours. The inhibition of ALK resulted in a significant decrease in NMN (FIG. 6B), which is a direct product of NAMPT, as well as its end product, NAD+(FIG. 6C). Additionally, they introduced NMN and NAD standards into the assay to validate the authenticity of the NMN and NAD metabolites identified in the study. To evaluate the effect of NAMPT Y188 phosphorylation on cell proliferation, the inventors cultured ALK+ALCL SUPM2 stable cell lines expressing NAMPT-HA-GFP-WT, NAMPT-HA-GFP-Y188F, and NAMPT-HA-GFP-SS-AA in a 96-well plate at 5×10³ cells/ml for 72 hours. Cell growth was monitored by measuring green fluorescence using a plate reader (N=6). The results showed a significant reduction in cell proliferation: more than 45% decrease (p<0.001) for cells expressing NAMPT-Y188F, and over 35% reduction (p>0.01) for those expressing NAMPT-SS-AA, compared to cells expressing NAMPT-WT (FIG. 6D). To assess the in vitro clonogenic potential of SUPM2 cells, the inventors conducted methylcellulose-based colony formation assays with cells expressing NAMPT-GFP-WT, NAMPT-GFP-Y188F, and NAMPT-GFP-SS-AA. These assays revealed a significantly higher number of colonies (over 70%, p<0.005) formed by cells expressing NAMPT-WT compared to those expressing NAMPT-Y188F and NAMPT-SS-AA (FIG. 6E, F). The inventors' working model suggests that NPM-ALK and other tyrosine kinases directly phosphorylate NAMPT Y188 to promote NMN and NAD+ biosynthesis, enhancing cellular proliferation.

### Example 19: Treatment of ALK+ALCL Cell Line SUPM2 with ALK Inhibitor, Certinib and NAMPT Inhibitor, FK866

The ALK+ALCL cell line SUPM2 was treated with ALK inhibitor, certinib and NAMPT inhibitor, FK866 for 24 hours and cell viability was measured by counting live cells using trypan blue exclusion method (FIG. 9, Panel A) and NAD content was measured using NAD/NADH assay kit (FIG. 9, Panel B) (Biovision Inc.) in biological triplicate. The difference between each group analyzed by using PRSIM Graph pad software and t-test p-values; 0.003, **; 0.0002, *** and 0.0001, ****.

NAMPT tyrosine phosphorylation on Y188 by NPM-ALK increases its enzymatic activity and inhibiting ALK activity by certinib inhibit cell viability by lowering NAD levels and impacting key metabolic enzymes. NAMPT is a rate limiting enzyme in NAD synthesis pathway, by inhibiting NAMPT tyrosine phosphorylation or activity inhibition by FK866 in SUPM2 cells regulate cell growth and NAD metabolism. The NAMPT inhibitor FK866 is commercially available for the research purpose and not approved for human use. The certinib ALK inhibitor is a FDA approved drug using for treating EML4-ALK positive non-small cell lung cancer (NSCLC), NPM-ALK+ALCL and neuroblastoma patients.

### Example 20: NPM-ALK Directly Phosphorylates the Critical NAMPT Residue Y188 and Regulates NAMPT Dimerization

HEK293T cells stably expressing HA-tagged NAMPT wild-type or SS-AA monomeric constructs were transfected in triplicate with empty vector or active NPM-ALK. NAMPT was immunoprecipitated using anti-HA agarose beads, resolved briefly by SDS-PAGE, stained with colloidal Coomassie Blue G-250, and analyzed by LC-MS/MS phosphoproteomics.

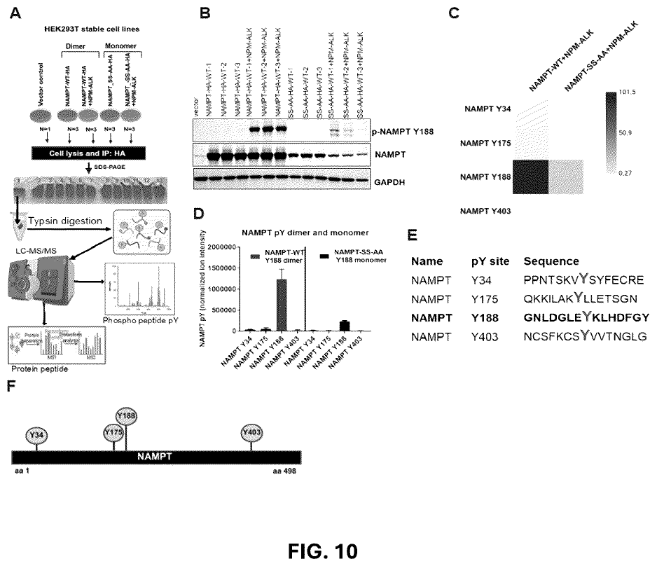

Phosphoproteomic analysis was performed to investigate tyrosine phosphorylation of NAMPT in the context of dimeric versus monomeric protein states. A schematic overview of the workflow illustrates the generation of HA-tagged NAMPT wild-type (WT; dimeric) and SS_AA mutant (monomeric) stable cell lines, followed by HA immunoprecipitation and LC-MS/MS analysis (FIG. 10, Panel A). Western blot analysis using a phospho-specific NAMPT Y188 antibody confirmed that NAMPT is phosphorylated on tyrosine residues in NPM-ALK-transfected cell lysates, validating Y188 as a bona fide phosphorylation site (FIG. 10, Panel B).

Phosphoproteomic profiling identified Y188 as the major tyrosine phosphorylation site on NAMPT. Heatmap analysis revealed a strong spectral intensity for phosphorylated Y188 in NAMPT-WT cells expressing active NPM-ALK, whereas this signal was markedly reduced in cells expressing the monomeric SS_AA mutant (FIG. 10, Panel C). Quantitative comparison of spectral intensities further demonstrated significantly higher tyrosine phosphorylation of NAMPT in the WT dimeric form compared with the monomeric mutant (FIG. 10, Panel D), indicating that NAMPT dimerization promotes efficient Y188 phosphorylation. Sequence analysis identified the tyrosine-phosphorylated residues and their surrounding motifs within NAMPT (FIG. 10, Panel E), and schematic mapping localized these residues within the NAMPT protein structure (FIG. 10, Panel F). Finally, sequence alignment across species showed that the Y188-containing region is highly conserved (FIG. 10, Panel G), suggesting an evolutionarily conserved and potentially functional role for Y188 phosphorylation. Collectively, these data indicate that NAMPT dimerization is critical for NPM-ALK-mediated phosphorylation at Y188 and support a conserved regulatory role for this modification.

In this study, we identify NAMPT Y188 as a major tyrosine phosphorylation site regulated by NPM-ALK and demonstrate that this modification is dependent on NAMPT dimerization. Phosphoproteomic analysis revealed robust Y188 phosphorylation in the dimeric NAMPT wild-type protein, whereas phosphorylation was markedly reduced in the monomeric SS-AA mutant, indicating that NAMPT structural integrity is critical for efficient tyrosine phosphorylation.

The dependence of Y188 phosphorylation on NAMPT dimerization suggests that dimer formation may facilitate kinase access or stabilize a conformation required for NPM-ALK-mediated phosphorylation. The high evolutionary conservation of the Y188 residue further supports a functional role for this site in NAMPT regulation. Given the established importance of NAMPT in cellular metabolism and cancer biology, phosphorylation at Y188 represents a previously unrecognized regulatory mechanism linking oncogenic tyrosine kinase signaling to metabolic control.

Collectively, these findings provide mechanistic insight into how NAMPT structure influences its post-translational regulation and suggest that NPM-ALK-driven phosphorylation of NAMPT may contribute to altered metabolic signaling in ALK-positive malignancies. Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating cancer in a subject in need thereof comprising administering to the subject a Nicotinamide Phosphoribosyltransferase (NAMPT) inhibitor.

2. The method according to claim 1, wherein the NAMPT inhibitor is a peptide.

3. The method according to claim 2, wherein the amino acid sequence of the peptide comprises PNTSKVYSYFECR (Pep-1; SEQ ID NO: 14), KLRKVKYEETVF (Pep-2; SEQ ID NO: 15), GWNYILEKYDGHL (Pep-3, SEQ ID NO: 16), KLHDFGYRGVSSQE (Pep-4, SEQ ID NO: 1), or KGDLEEYGQDL (Pep-5, SEQ ID NO: 17).

4. The method according to claim 3, wherein the amino acid sequence of the peptide comprises PNTSKVYSYFECR (Pep-1; SEQ ID NO: 14), GWNYILEKYDGHL (Pep-3, SEQ ID NO: 16), or KLHDFGYRGVSSQE (Pep-4, SEQ ID NO: 1).

5. The method according to claim 4, wherein the amino acid sequence of the peptide comprises KLHDFGYRGVSSQE (Pep-4, SEQ ID NO: 1).

6. The method according to claim 1, wherein the NAMPT inhibitor is a small molecule.

7. The method according to claim 6, wherein the small molecule is daporinad, GMX1777, GMX1778, KPT-9274, or OT-92.

8. The method according to claim 1, wherein the cancer is ALK+ anaplastic large cell lymphoma (ALCL), EML4-ALK positive non-small cell lung cancer (NSCLC), or breast cancer with the activated form of the type 1 insulin-like growth factor receptor (IGF1R).

9. The method according to claim 1, the method further comprising administering a second therapeutic agent selected from the group consisting of an anaplastic lymphoma kinase (ALK) inhibitor, a bruton tyrosine kinase (BTK) inhibitor, a spleen tyrosine kinase (SYK) inhibitor, or any combination thereof.

10. The method according to claim 9, wherein the ALK inhibitor is certinib.

11. The method according to claim 1, wherein the NAMPT inhibitor is administered to the subject intravenously.

12. A method of diagnosing a cancer in a subject comprising detecting the presence of Y188 tyrosine phosphorylation of Nicotinamide Phosphoribosyltransferase (NAMPT) in a cell obtained from the subject.

13. The method according to claim 12, wherein the cancer is anaplastic large cell lymphoma (ALCL), non-Hodgkin's lymphoma, pediatric non-Hodgkin's lymphoma, EML4-ALK positive non-small cell lung cancer (NSCLC), or breast cancer with the activated form of the type 1 insulin-like growth factor receptor (IGF1R).

14. The method according to claim 13, wherein the cell obtained from the subject is a cell from a lymph node, lung, or breast.

15. The method of claim 12, wherein cancer is breast cancer.

16. A method of treating diabetes, obesity, or a neurodegenerative disease in a subject in need thereof comprising administering to the subject a Nicotinamide Phosphoribosyltransferase (NAMPT) activator.

17. The method according to claim 16, wherein the NAMPT activator is SBI-797812 or P7C3.

18. The method according to claim 16, wherein the NAMPT inhibitor is administered to the subject intravenously.

19. The method according to claim 16, wherein the NAMPT activator is SBI-797812 and the disorder comprises diabetes.

20. The method according to claim 16, wherein the NAMPT activator is SBI-797812 and the disorder comprises neurodegenerative disease.


SEQUENCE LISTING (21 sequences)

SEQ ID NO: 1 - protein, 14 aa, Homo sapiens
KLHDFGYRGVSSQE

SEQ ID NO: 2 - protein, 16 aa, Homo sapiens
QYPPNTSKVYSYFECR

SEQ ID NO: 3 - protein, 18 aa, Homo sapiens
KVKYEETVFYGLQYILNK

SEQ ID NO: 4 - protein, 20 aa, Homo sapiens
IQEAKDVYKEHFQDDVFNEK

SEQ ID NO: 5 - protein, 8 aa, Homo sapiens
GWNYILEK

SEQ ID NO: 6 - protein, 10 aa, Homo sapiens
YDGHLPIEIK

SEQ ID NO: 7 - protein, 22 aa, Homo sapiens
YLLETSGNLDGLEYKLHDFGYR

SEQ ID NO: 8 - protein, 27 aa, Homo sapiens
LHDFGYRGVSSQETAGIGASAHLYNFK

SEQ ID NO: 9 - protein, 12 aa, Homo sapiens
KFPVTENSKGYK

SEQ ID NO: 10 - protein, 16 aa, Homo sapiens
GDLEEYGQDLLHTVFK

SEQ ID NO: 11 - protein, 8 aa, Homo sapiens
SYSFDEIR

SEQ ID NO: 12 - protein, 15 aa, Homo sapiens
QKKILAKYLLETSGN

SEQ ID NO: 13 - protein, 15 aa, Homo sapiens
NCSFKCSYVVTNGLG

SEQ ID NO: 14 - protein, 13 aa, Homo sapiens
PNTSKVYSYFECR

SEQ ID NO: 15 - protein, 12 aa, Homo sapiens
KLRKVKYEETVF

SEQ ID NO: 16 - protein, 13 aa, Homo sapiens
GWNYILEKYDGHL

SEQ ID NO: 17 - protein, 11 aa, Homo sapiens
KGDLEEYGQDL

SEQ ID NO: 18 - protein, 15 aa, Homo sapiens
PPNTSKVYSYFECRE

SEQ ID NO: 19 - protein, 15 aa, Homo sapiens
GNLDGLEYKLHDFGY

SEQ ID NO: 20 - protein, 13 aa, Homo sapiens
  MOD_RES 7: note = phosphorylation
NLDGLEYKLHDFG

SEQ ID NO: 21 - protein, 7 aa, Homo sapiens
GLEYKLH